(19) United States
(12) Patent Application Publication
(10) Pub. No.: US 2008/0255501 A1
(43) Pub. Date: Oct. 16, 2008

(54) PERCUTANEOUS DELIVERY AND RETRIEVAL SYSTEMS FOR SHAPE-CHANGING ORTHOPEDIC JOINT DEVICES

(76) Inventors: Michael Hogendijk, Mountain View, CA (US); Janine Robinson, Half Moon Bay, CA (US); David White, Atherton, CA (US); Michael James Orth, Morgan Hill, CA (US)

Correspondence Address: MORRISON & FOERSTER LLP, 755 PAGE MILL RD, PALO ALTO, CA, 94304-1018, US

(21) Appl. No.: 12/099,296

(22) Filed: Apr. 8, 2008

Related U.S. Application Data
(60) Provisional application No. 60/911,056, filed on Apr. 10, 2007.
(60) Provisional application No. 60/975,444, filed on Sep. 26, 2007.

(51) Int. Cl. A61M 31/00; A61B 19/00

(52) U.S. Cl. 604/64; 604/60; 128/898

(57) ABSTRACT

Delivery and retrieval systems for delivering or retrieving a shape-changing percutaneously implantable orthopedic device joint prosthesis that can move between a generally arcuate configuration and a substantially straightened or slightly curved configuration. The orthopedic device can be delivered with a needle that is joint expanding, actuating, pivotable, or can include a balloon. The orthopedic device acts as a soft compliant bearing surface or cushion that minimizes the bone-on-bone wear from articulation and loading and may include a covering or coating with tissue or an expanding hydrophilic material. The orthopedic device delivery system can include a loading device with a channel for storing the orthopedic device in a non-straightened configuration and orienting the orthopedic device in the proper implantation orientation. The orthopedic device can be advanced or retrieved through the loading device with a knob and/or a flexible plunger.

### CROSS-REFERENCE TO RELATED APPLICATIONS

This application claims the benefit of priority from U.S. Provisional No. 60/911,056 entitled “PERCUTANEOUSLY DELIVERABLE ORTHOPEDIC JOINT DEVICE” filed Apr. 10, 2007, and U.S. Provisional No. 60/975,444 entitled “PERCUTANEOUS DELIVERY AND RETRIEVAL SYSTEMS FOR SHAPE-CHANGING ORTHOPEDIC JOINT DEVICES WITH A CASSETTE” filed Sep. 26, 2007, each of which are incorporated in their entirety by reference, herein.

### BACKGROUND

Various embodiments of the present inventions relate to the treatment of osteoarthritis, rheumatoid arthritis, and any other joint degenerative process with a minimally invasive implantable device to reduce, amongst other things, bone-to-bone contact at a joint.

Today there are an increasing number of patients with osteoarthritis, rheumatoid arthritis, and other joint degenerative processes. Osteoarthritis is by far the most common type of arthritis, and the percentage of people who have it grows higher with age. An estimated 12.1 percent of the U.S. population (nearly 21 million Americans) age 25 and older have osteoarthritis of one form or another. Although more common in older people it usually is the result of a joint injury, a joint malformation, or a genetic defect in joint cartilage. Its time of occurrence differs: osteoarthritis tends to start for men before the age of 45, and after the age of 45 it is more common in women. It is also more likely to occur in people who are obese or overweight and is related to those jobs that stress particular joints.

It affects the musculoskeletal system and specifically the joints—where two or more bones meet. It most often occurs in the hands (particularly at the ends of the fingers and thumbs, between phalanges, metacarpals and/or carpals), feet (in the toes, between phalanges, metatarsals and/or tarsals), wrists, elbows, shoulders, knees, hips, and the spine (particularly at the neck and lower back). Joint problems can include; stiffness, inflammation and damage to joint cartilage (the tough, smooth tissue that covers the ends of the bones, enabling them to glide against one another) and surrounding structures. Such damage can lead to joint weakness, instability and visible deformities that, depending on the location of joint involvement, can interfere with the most basic daily tasks such as walking, climbing stairs, using a computer keyboard, cutting your food or brushing your teeth. This ultimately results in moderate to severe pain and joint deterioration. As this is a degenerative process of the joint it can ultimately end in total joint replacement. Drug regimes can provide temporary relief from the pain but do not slow down the crippling affects. The extreme result or end point in traditional treatments is an open surgery procedure for placing a spacer or total joint replacement with a prosthetic device. It would be desirable as well as beneficial if there were an intermediary step or alternative treatment before this extreme.

Current joint replacement therapies (spacers or a total prosthesis) require the joint capsule to be surgically opened and the bone surfaces to be partially or totally removed. Various spacers and or prosthetic devices can be made from a number of biocompatible polymers such as silicone, polyurethane, Teflon etc. Both modalities present drawbacks. For example, U.S. Pat. No. 6,007,580 to Matti Lehto et al. describes an implantable spacer that must be fixed at one or both ends to the bone of either end of the knuckle. It is not provided in a shape memory configuration and must be implanted by opening of the knuckle capsule. It further must be affixed at one or both ends to the corresponding bone faces.

Various spacers in the art can cause inflammation and the total joint replacement can limit the range of motion, compromise the strength of and ultimately the stability of the joint. These surgeries are invasive and require the joint capsule to be surgically opened. The incision itself can result in inflammation and infection. Due to the invasiveness of the procedure and the delicate nature of the joint it can result in joint instability prolonged healing times.

### SUMMARY

It would be desirable to provide a supplemental or alternative form of treatment that could be provided before the more drastic step of total joint replacement. Such intermediary treatment preferably comprises providing an embodiment of an orthopedic device comprising a biocompatible cushion or improved spacer made of shape-changing, shape-memory or shape-recovering material placed into the joint to minimize pain and slow the deterioration process. In one embodiment the orthopedic device would be sized to preserve a proper, natural space, distance, or gap between bones in a joint for proper articulation of the bones in the joint. The characteristics of the orthopedic device in terms of at least thickness, width and/or diameter, configuration in one or more planes, flexibility, deflection in response to joint type (degrees of freedom, types of tissue present, size of a joint capsule if present, etc.) size, and/or type of movement (such as articulation and/or compression) may be configured or selected based on the characteristics of the joint and the patient receiving the orthopedic device. In various embodiments, characteristics of the orthopedic implant could be configured for implantation in an infant to a large patient, such as an athlete. Certain embodiments can be configured with characteristics and dimensions for implantation in a patient that is an animal, ranging is sizes from small to large, including but not limited to mammals such as mice, dogs, cats and others. It would further be desirable to provide this cushion or improved articulation device in a minimally-invasive procedure; e.g., through a tubular delivery apparatus such as a hypodermic needle, cannula or catheter with a lumen that can be inserted directly into the joint without the necessity of a surgical cut-down procedure and its associated risks. There would be a distinct benefit to the patient in that there would be a reduction in pain, time, and complexity in conducting the procedure as well as decreasing healing time, reducing post-operative pain, and slowing of deterioration in a joint without the necessity of surgically opening the joint. In certain embodiments the orthopedic device can have a coating or covering that is made of tissue, a joint or external fluid-expanding material, a hydrophilic material, or other material.

In various embodiments the orthopedic device is an implantable, biocompatible prosthetic generally arcuate open ring, open hoop, open loop or spiral which is delivered through a hypodermic needle in a narrowed configuration or a substantially straightened configuration and into the joint. Then due to its shape memory set, it then assumes an open ring. This ring acts as a compliant bearing surface which minimizes the bone on bone contact and wear from articulation and loading. In another embodiment the orthopedic device is an implantable prosthetic generally rectilinear polygon, an open polygon, or series of linear segment shapes which is delivered through a hypodermic needle in a narrowed configuration or a substantially straightened configuration and into the joint.

In one embodiment the orthopedic device is an implantable prosthetic with a series of discrete articulatable elements. The elements, or segments, can be connected by one or more connectors. In one embodiment the orthopedic device is a ratcheted linkage. In another embodiment the orthopedic device is a series of articular layers on a bendable elongate core. In one embodiment the orthopedic device discrete articulatable elements can form a generally arcuate open ring or spiral. In various embodiments the orthopedic device may be delivered through a hypodermic needle in a narrowed configuration or a substantially straightened configuration and into the joint. After delivery, various embodiments of the orthopedic device can resume it generally rectilinear or generally arcuate configuration by being manipulated into that shape or due to a shape memory set. The orthopedic device can act as a compliant bearing surface which minimizes the bone on bone wear from articulation and loading.

In various embodiments, delivery or retrieval systems include a straight or curved tubular delivery apparatus such as a hypodermic needle, syringe, cannula or catheter with a lumen specially configured to implant or retrieve an orthopedic device with a specific orientation. Certain systems can include specially shaped plungers, needles (such as expandable, pivotable, or balloon expansion needles), interlocks, removable attachments, pinchers, lassos, tethers, hooks, threaded interfaces, reservoirs, or cassette loading systems for interacting with or positioning the orthopedic device. In one embodiment the orthopedic device is an implantable prosthetic generally arcuate open ring or spiral which is delivered through a hypodermic needle in a narrowed configuration or a substantially straightened configuration and into the joint. Then due to its shape memory set, it then assumes an open ring. This ring acts as a compliant bearing surface which minimizes the bone on bone wear from articulation and loading. In another embodiment the orthopedic device is an implantable prosthetic generally rectilinear polygon, open polygon, or series of linear segment shapes that is delivered through a hypodermic needle in a narrowed configuration or a substantially straightened configuration and into the joint.

In one preferred embodiment a cassette system for storing orthopedic devices in an arcuate or rectilinear, non-straightened configuration can be used in an orthopedic device delivery system. In various embodiments, one or more orthopedic devices can be advanced through a cassette with a barrel, groove, knob, and/or flexible plunger system for advancing or retrieving the implants.

### BRIEF DESCRIPTION OF THE DRAWINGS

These and other features, embodiments, and advantages of the present invention will now be described in connection with preferred embodiments of the invention, in reference to the accompanying drawings. The illustrated embodiments, however, are merely examples and are not intended to limit the invention.

FIG. 1A is a schematic top view of an orthopedic device according to one embodiment of the present invention comprising a substantially straightened configuration.

FIG. 1B is a schematic top view of an orthopedic device according to one embodiment of the present invention comprising an open hoop arcuate configuration.

FIG. 1C is a schematic top view of an orthopedic device according to one embodiment of the present invention comprising a nautilus-style spiral arcuate configuration.

FIG. 2 is a schematic cross-section view perpendicular to a longitudinal axis of an orthopedic device according to one embodiment of the present invention comprising an elongate core and an articular layer surrounding at least a portion of the core.

FIG. 3A is a schematic cross-section view along a plane substantially parallel to and passing through a longitudinal axis of an orthopedic device according to one embodiment of the present invention comprising a substantially straightened configuration, the device comprising an elongate core and an articular layer surrounding at least a portion of the core.

FIG. 3B is a schematic cross-section view along a plane substantially parallel to and passing through a longitudinal axis of an orthopedic device according to one embodiment of the present invention comprising an open hoop arcuate configuration, the device comprising an elongate core and an articular layer surrounding at least a portion of the core.

FIG. 3C is a schematic cross-section view along a plane substantially parallel to and passing through a longitudinal axis of an orthopedic device according to one embodiment of the present invention comprising a nautilus-style spiral arcuate configuration, the device comprising an elongate core and an articular layer surrounding at least a portion of the core.

FIG. 3D is a schematic cross-section view along a plane substantially parallel to and passing through a longitudinal axis of an orthopedic device according to one embodiment of the present invention comprising an open hoop arcuate configuration, the device comprising one or more elongate cores wrapped, braided or folded along a length of the device and an articular layer surrounding at least a portion of the core.

FIG. 3E is a schematic cross-section view along a plane substantially parallel to and passing through a longitudinal axis of an orthopedic device according to one embodiment of the present invention comprising a nautilus-style spiral arcuate configuration, the device comprising one or more elongate cores wrapped, braided or folded along a length of the device and an articular layer surrounding at least a portion of the core.

FIG. 4A is a schematic side view of an elongate core according to one embodiment of the present invention comprising one or more substantially linear or straight members.

FIG. 4B is a schematic side view of an elongate core according to one embodiment of the present invention comprising one or more wave, curve or zig-zag members disposed in one or more planes.

FIG. 4C is a schematic side view of an elongate core according to one embodiment of the present invention comprising one or more members in a braided or weave configuration.

FIG. 5A is a schematic top view of an elongate core according to one embodiment of the present invention comprising an open hoop arcuate configuration and one or more end pieces.

FIG. 5B is a schematic top view of an elongate core according to one embodiment of the present invention comprising an open hoop arcuate configuration and one or more bends or hooks.

FIG. 5C is a schematic top view of an elongate core according to one embodiment of the present invention comprising an open hoop arcuate configuration and one or more features bent in or out of the primary plane of the device.

FIG. 5D is a schematic side view of an orthopedic device according to one embodiment of the present invention comprising a multiplanar spiral configuration.

FIG. 5E is a schematic side view of an orthopedic device according to one embodiment of the present invention comprising a multiplanar arcuate configuration.

FIG. 5F is a schematic side view of an orthopedic device according to one embodiment of the present invention comprising a “W”-shaped configuration.

FIGS. 6A-6K are schematic cross-section views of elongate cores according to various embodiments of the present invention.

FIG. 7A is a schematic perspective view of an orthopedic device according to one embodiment of the present invention comprising a plurality of independent or interconnectable discrete elongate members.

FIG. 7B is a schematic perspective view of an orthopedic device according to one embodiment of the present invention comprising a plurality of independent or interconnectable discrete elongate members in a “W” configuration.

FIG. 8 is a schematic perspective view of an orthopedic device according to one embodiment of the present invention comprising a plurality of independent or interconnectable discrete members.

FIG. 9A is a schematic side view of an elongate core according to one embodiment of the present invention comprising a plurality of interconnectable discrete members in a substantially straightened configuration.

FIG. 9B is a schematic side view of one interconnectable discrete member of FIG. 9A.

FIG. 9C is a schematic side view of an elongate core comprising a plurality of interconnectable discrete members according to FIG. 9A in an arcuate open loop configuration.

FIG. 10A is a schematic side view of an orthopedic device delivery system according to one embodiment of the present invention comprising a handle and a plunger.

FIG. 10B is a schematic side view of an orthopedic device delivery system according to one embodiment of the present invention comprising a substantially straight cannula or needle with a lumen.

FIG. 10C is a schematic side view of an orthopedic device delivery system according to one embodiment of the present invention comprising an arcuate cannula or needle with a lumen.

FIG. 10D is a schematic side view close up of a distal end of an orthopedic device delivery system according to one embodiment of the present invention comprising a blunted delivery cannula.

FIG. 10E is a schematic side view of an orthopedic device delivery system according to one embodiment of the present invention comprising an angular tip.

FIG. 11 is a schematic side view of an orthopedic device delivery system according to one embodiment of the present invention comprising an implantable orthopedic device, a cannula, and a plunger.

FIG. 12A is a schematic side cross-sectional view of an orthopedic device delivery system according to one embodiment of the present invention prior to implantation in a joint.

FIG. 12B is a schematic top cross-sectional view orthogonal to FIG. 12A of two embodiments of orthopedic device delivery systems similar to the system of FIG. 12A prior to implantation in a joint, wherein on embodiment comprises a substantially straight cannula and the other embodiment comprises an arcuate cannula.

FIG. 13A is a schematic side cross-sectional view of an orthopedic device delivery system according to the embodiment of the present invention shown in FIG. 12A upon partial insertion of the orthopedic device into the joint.

FIG. 13B is a schematic top cross-sectional view orthogonal to FIG. 13A of two embodiments of orthopedic device delivery systems according to FIG. 12B upon partial insertion of the orthopedic device into the joint.

FIG. 14A is a schematic side cross-sectional view of an orthopedic device delivery system according to the embodiment of the present invention shown in FIG. 12A upon deployment of the orthopedic device into the joint.

FIG. 14B is a schematic top cross-sectional view orthogonal to FIG. 14A of two embodiments of orthopedic device delivery systems according to FIG. 12B upon deployment of the orthopedic device into the joint.

FIG. 15A is a schematic side cross-sectional view of an orthopedic device delivery system according to the embodiment of the present invention shown in FIG. 12A upon deployment of the orthopedic device into the joint and removal of the delivery cannula.

FIG. 15B is a schematic top cross-sectional view orthogonal to FIG. 15A of two embodiments of orthopedic device delivery systems according to FIG. 12B upon deployment of the orthopedic device into the joint and removal of the delivery cannula(e).

FIG. 16A is a schematic side view of an orthopedic device according to one embodiment of the present invention comprising a tether and a loop structure in a substantially straightened configuration.

FIG. 16B is a schematic side view of the orthopedic device of FIG. 16A in an arcuate configuration.

FIG. 16C is a schematic side view of an orthopedic device according to one embodiment of the present invention comprising one or more tethers in an arcuate configuration.

FIG. 17 is a schematic side view of an orthopedic device according to one embodiment of the present invention comprising a looped arcuate configuration and at least one anchor.

FIG. 18 is a schematic side view of an orthopedic device removal system according to one embodiment of the present invention comprising an implantable orthopedic device, a cannula, and a snare.

FIGS. 19A and 19B are schematic perspective and side views of a portion of an interface in an orthopedic device delivery and removal system according to one embodiment of the present invention comprising an implantable orthopedic device and a plunger connectable with a device interface.

FIGS. 20A-20C are schematic side views of a portion of an interface in an orthopedic device delivery and removal system according to another embodiment of the present invention comprising an implantable orthopedic device and a plunger connectable with a device interface.

FIG. 21A is a schematic perspective view of an orthopedic device delivery system according to one embodiment of the present invention comprising a loading device for storing the orthopedic device in an arcuate configuration.

FIG. 21B is a schematic side view of the orthopedic device delivery system comprising a loading device of FIG. 21A.

FIG. 21C is a schematic side view of an orthopedic device delivery system according to one embodiment of the present invention comprising a loading device comprising a needle and a loop for storing the orthopedic device in an arcuate configuration.

FIG. 22A is a schematic side view of an orthopedic device delivery system according to one embodiment of the present invention comprising a loading device cassette and a cannula or needle with a channel.

FIG. 22B is a schematic side view of the orthopedic device delivery system of FIG. 22A with an orthopedic device being advanced from the loading device cassette and into the lumen of the cannula or needle.

FIG. 23 is a schematic perspective view of an orthopedic device delivery system according to one embodiment of the present invention comprising a cassette and a needle with a lumen.

FIG. 24 is a schematic side view of an orthopedic device delivery system according to one embodiment of the present invention comprising a plunger.

FIG. 25 is a schematic perspective view of an orthopedic device delivery system according to one embodiment of the present invention comprising a cassette barrel with an orthopedic device groove.

FIG. 26 is a schematic side view of an orthopedic device delivery system according to one embodiment of the present invention comprising a cassette barrel with an orthopedic device groove.

FIG. 27A is a partial cut-away schematic side view of an orthopedic device delivery system according to one embodiment of the present invention comprising a cassette, barrel and plunger.

FIG. 27B is a partial cut-away schematic side view of an orthopedic device delivery system according to one embodiment of the present invention comprising a cassette, barrel and plunger.

FIGS. 28A-28E are partially exploded cut-away schematic side views of an orthopedic device delivery system according to one embodiment of the present invention comprising a cassette, barrel and plunger.

FIGS. 29A and 29B are schematic perspective views of one embodiment of a needle with an expandable distal tip for orthopedic device delivery.

FIGS. 30A and 30B are schematic side views of one embodiment of a balloon to assist in orthopedic device delivery.

FIGS. 31A-31D are partial cut-away schematic side views of an orthopedic device delivery system according to one embodiment of the present invention comprising a plunger, a loading device and a cannula.

FIG. 32A is a schematic rear view orthogonal to FIG. 31A of an embodiment of a knob configured to work with the loading device of the embodiment of the orthopedic device delivery system of FIG. 31A.

FIG. 33A is a schematic side view of an embodiment of a knob configured to work with the loading device of the embodiment of the orthopedic device delivery system of FIG. 31A.

FIGS. 34A-34C are partial cut-away schematic side views of an orthopedic device delivery system according to one embodiment of the present invention comprising a cannula, a loading device, and a handle with a pistol grip configuration.

FIGS. 35A-35C are partial cut-away schematic side views of an orthopedic device delivery system according to one embodiment of the present invention comprising a cannula, a loading device, a delivery knob, and a handle.

FIG. 36A is a schematic front view orthogonal to FIG. 35A of the orthopedic device delivery system of FIG. 35A.

FIG. 36C is a partial cut-away schematic front view orthogonal to FIG. 35A of the orthopedic device delivery system of FIG. 35C.

FIGS. 37A-37C are partial cut-away schematic side views of an orthopedic device delivery system according to one embodiment of the present invention comprising a cannula, a loading device, a handle and a finger-loop trigger.

FIGS. 38A-38C are partial cut-away schematic side views of an orthopedic device delivery system according to one embodiment of the present invention comprising a cannula, a loading device, a proximal delivery knob and a handle.

FIGS. 39A-39B are partial cut-away schematic side views of an orthopedic device delivery system according to one embodiment of the present invention comprising a cannula, a loading device, a delivery knob and a handle.

FIGS. 40A-40C are partial cut-away schematic side views of an orthopedic device delivery system according to embodiments of the present invention comprising a cannula, a handle and a push-button actuated push rod.

FIGS. 41A-41C are partial cut-away schematic bottom views of an orthopedic device delivery system according to one embodiments of the present invention comprising a cannula, a handle a loading device and a removable tissue piercing device.

Throughout the figures, the same reference numerals and characters, unless otherwise stated, are used to denote like features, elements, components or portions of the illustrated embodiments. In certain instances similar reference number schemes are used whereby the reference numerals referred to as “AA” in reference numeral “AAxx” correspond to a figure while the “xx” is directed to similar or interchangeable features, elements, components or portions of the illustrated embodiments in different figures. In certain instances, similar names may be used to describe similar components with different reference numerals which have certain common or similar features. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF THE PREFERRED EMBODIMENTS

As should be understood in view of the following detailed description, this application is primarily directed to apparatuses, systems and methods for minimally-invasive treatment of bone joints. Bone joints contemplated for various embodiments of the orthopedic device include, but are not limited to, hands (fingers and thumbs, between phalanges, metacarpals and/or carpals), feet (in the toes, between phalanges, metatarsals and/or tarsals), wrists, elbows, shoulders, knees, hips, and the spine (particularly at the neck and lower back). In various embodiments, an orthopedic device suitable for minimally invasive deployment using a tubular delivery apparatus with a lumen or channel, such as a cannula, hypodermic needle, catheter, or another similar apparatus, any of which can be used interchangeably with each other in various embodiments. In one preferred embodiment of the invention, an orthopedic device comprises an elongate shape memory body that has a generally arcuate configuration to enhance self-centering positioning of the orthopedic device when deployed. In another embodiment an orthopedic device comprises an elongate shape memory body that has a generally rectilinear configuration to enhance self-centering positioning of the orthopedic device when deployed. In one embodiment an orthopedic device comprises a plurality of elongate shape memory bodies that can be moved into a configuration to enhance self-centering positioning of the orthopedic device when deployed. The body can be manipulated into a substantially straightened configuration to permit delivery. In various embodiments, the orthopedic device can be for single or multiple uses, and may be removed from the joint.

1. Implantable Orthopedic Devices

In various embodiments the orthopedic device can have an arcuate configuration once it is implanted in a joint. As used herein, “arcuate” may refer to curved or rounded configurations or shapes, but can also include generally arcuate configurations and shapes that have some straight aspect or element with curved or rounded configurations or shapes. As used herein, arcuate and generally arcuate shapes can include “C”, “O”, “S”, spiral, nautilus, “Q” and other generally arcuate shapes which can be planar or non-planar. Similarly, certain embodiments of the orthopedic device may include rectilinear configurations, which can include polygons such as triangles, squares, rectangles, diamonds, rhombuses, pentagons, hexagons, octagons and other shapes with generally straight edges, and further including shapes and configurations that are generally rectilinear having some curved edge or corners or segments among rectilinear shapes. As used herein, rectilinear and generally rectilinear shapes can include “N”, “M”, “W”, “Z”, “T”, “Y”, “V”, “L”, “X” and other generally rectilinear shapes. Various embodiments of generally arcuate or generally rectilinear shapes can include shapes with both rectilinear and arcuate portions, such as a “P”, “R”, “B”, and “U”. Embodiments of the orthopedic device have three major dimensions, which can correspond to a first major dimension, a second major dimension and a third major dimension. In one embodiment the first major dimension, second major dimension and third major dimension correspond to a width, a height and a thickness. Certain embodiments of the orthopedic device have a thickness which corresponds to the smallest dimension, which roughly correspond to fit in the space between articulating surfaces of tissue such as bone or cartilage in a joint. In one embodiment the width and height can be the same, such as with a circular or square shaped orthopedic device, or the height and width may be different as with an oval shape or a rectangle or other shape with non-equal height and width. In various embodiments the orthopedic implant can be implanted in joints of varying sizes in which the first major dimension and second major dimension may have a range of roughly 0.0394 to 4.0 inches (1.0-101.6 mm) and the third major diameter may have a range of roughly 0.001-0.50 inches (0.025-12.7 mm).

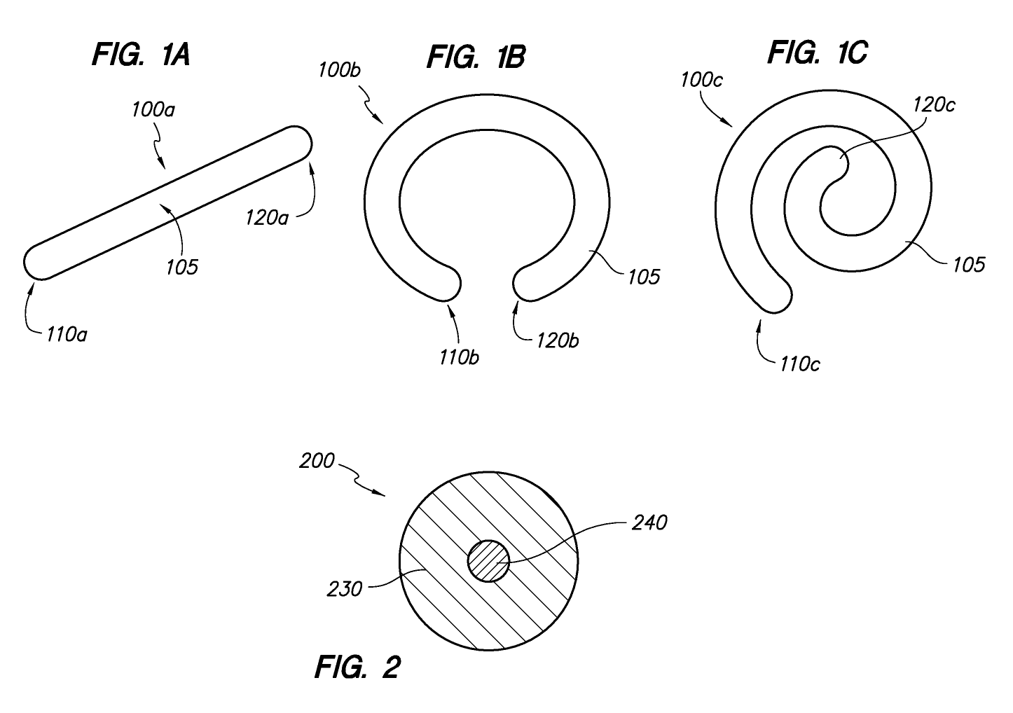

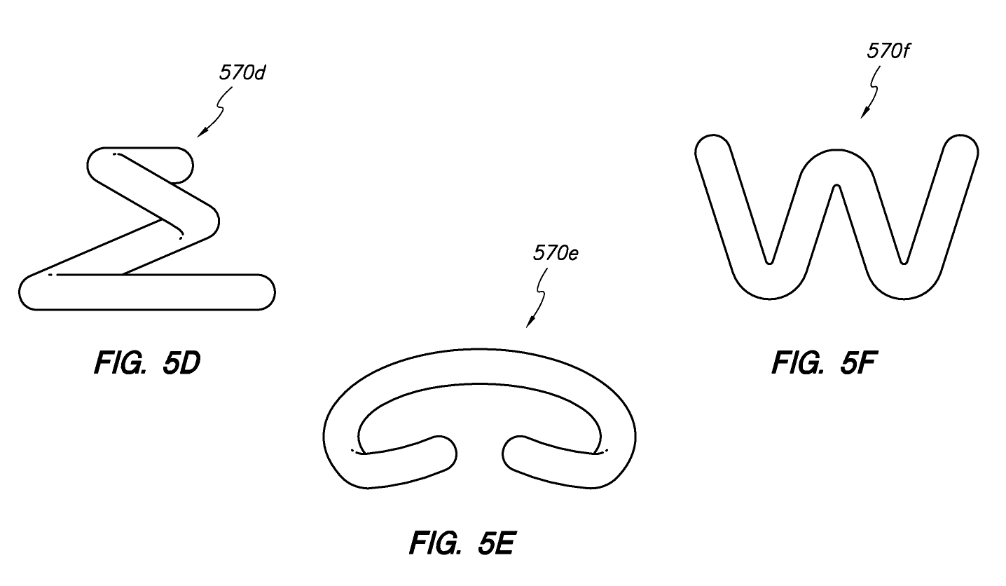

In order to deliver certain embodiments of the orthopedic device to a joint, various contemplated embodiments of delivery systems manipulate the shape of the orthopedic device into a narrowed configuration to fit in a lumen of a delivery tube or delivery device. In one embodiment, a narrowed configuration comprises the reduction of the first major dimension. In one embodiment, a narrowed configuration comprises the reduction of the second major dimension. In one embodiment, a narrowed configuration comprises the reduction of the third major dimension. In one embodiment, a narrowed configuration comprises a combination of the reduction of the first major dimension, second major dimension and/or the third major dimension. In some narrowing configuration embodiments, certain major dimensions are reduced while others are increased. In one embodiment the orthopedic device can be moved into a narrowed configuration comprising pinching or narrowing the device so that parts of the orthopedic device overlap, such as with a C-shape being collapsed into an alpha shape (α), a gamma shape (γ), a twisted shape, a helix, and/or a multiplanar configuration such as illustrated in one embodiment at FIGS. 5B and 5C. In one embodiment the orthopedic device can be moved into a straightened or a substantially straightened configuration. In one embodiment the orthopedic device can be completely straightened (e.g. moved into a linear configuration). In one embodiment the orthopedic device may have a substantially straightened configuration, which includes a completely straightened, linear configuration as well as configurations in which at least a part of the orthopedic device is straightened or partially straightened, configurations in which arcuate orthopedic devices can be made less-arcuate and configurations in which rectilinear orthopedic devices can be made less-rectilinear. In one embodiment, an orthopedic device can be curved in an arcuate configuration that is less-curved, or has a larger major diameter, than the device as fully deployed in the joint. For example, FIG. 1A shows one embodiment of an orthopedic device 100a with substantially straightened configuration. The orthopedic device 100a has a proximal end 110a and a distal end 120a in relation to insertion into the body of a patient, such as into a joint. In various embodiments of orthopedic devices discussed herein, the distal end of the orthopedic device is advanced or inserted into the body of a patient first, while the proximal end of the orthopedic device is initially inserted proximal to the distal end. In various embodiments, the orthopedic device 100a has various shape configurations to permit loading from a lumen within a needle, cannula, or other device for delivering the orthopedic device to the site for implantation. In one embodiment the straightened configuration of orthopedic device 100a is suited for delivery from a substantially straight needle. In other embodiment configurations, the orthopedic device 100a is flexible and can be bent or biased to have a curve or other shape to permit delivery from curved or other-shaped needles or cannulae. In one embodiment the orthopedic device 100a is delivered over a delivery structure.

As illustrated, one embodiment of the orthopedic device has a relatively consistent width of the elongate device. However, in other contemplated embodiments, the width of the device body can vary along its length. For example, the orthopedic device can have a taper along a portion of its length, or be tapered along the device's entire length. Width, or other dimension, can vary from large to small or small to large, making the device thicker in some portions than in others. In one embodiment the device can be radially compressed along part or over the entire length of the device. In one embodiment the device can be compressed such that its cross section is reduced to a smaller cross section, so that for example, the device could come out of a delivery system and expand in its cross section. In one embodiment the device can be axially compressed or axially stretched along part or over the entire length of the device.

In one embodiment, the orthopedic device 100a is made of a shape memory material. For example, the shape memory material can be made from a heat set/shaped shape-memory material, such as Nitinol or a shape memory plastic, polymeric, or synthetic material, such as polycarbonate urethane. For example, one embodiment of the orthopedic device 100a comprises a shape memory material including a shape memory polyurethane or polyurethane-urea polymer. One example of this type of shape memory material is described in United States Patent Publication 2002/0161114 A1 entitled “Shape memory polyurethane or polyurethane-urea polymers” that is incorporated in its entirety by reference herein. Publication 2002/0161114 A1 describes a shape memory polyurethane or polyurethane-urea polymer including a reaction product of: (A) (a) silicon-based macrodiol, silicon-based macrodiamine and/or polyether of the formula (I): A—[(CH₂)ₘ—O—]ₙ—(CH₂)ₘ—A′, wherein A and A are endcapping groups; m is an integer of 6 or more; and n is an integer of 1 or greater; (b) a diisocyanate; and (c) a chain extender; or (B) (b) a diisocyanate: and (c) a chain extender, said polymer having a glass transition temperature which enables the polymer to be formed into a first shape at a temperature higher than the glass transition temperature and maintained in said first shape when the polymer is cooled to a temperature lower than the glass transition temperature, said polymer then being capable of resuming its original shape on heating to a temperature higher than the glass transition temperature. Various embodiments of the present invention relate to a shape memory polymer alone or a shape memory composition which includes a blend of two or more of the shape memory polyurethane or polyurethane-urea polymers defined above or at least one shape memory polyurethane or polyurethane-urea polymer defined above in combination with another material. The present invention further relates to processes for preparing materials having improved mechanical properties, clarity, processability, biostability and/or degradation resistance and devices or articles containing the shape memory polyurethane or polyurethane-urea polymer and/or composition defined above.

In one embodiment the orthopedic device 100a comprises an articular layer 105, which may also be called a blanket or a jacket. The articular layer 105 is sized and configured to be placed within a body, such as in a joint as a layer between bones of the joint to provide a slideable articulation surface and/or a cushion. In various embodiments the articular layer can range from 0.001 inches thick to 0.5 inches thick (0.025 mm-12.7 mm). In one embodiment the articular layer 105 is configured to be compressed by loading in the joint. For example, in one embodiment an articular layer may be compressed from a substantially circular cross-sectional shape to an oval, elliptical, or football shaped cross-section, which further increases the amount of surface coverage of the articular layer with respect to bony joint contact, resulting in reduced pressure at the joint. In one embodiment the operating range of compression of an orthopedic device is in the range of 0 to 50% of the cross sectional diameter.

In one embodiment the articular layer can be at least partially attached to the outside of a portion of a backbone or core. In one embodiment the articular layer can be attached to the outside of a portion of a backbone or core prior to implantation. In one embodiment the articular layer can be attached to the outside of a portion of a backbone or core during or after implantation. For one non-limiting example, a core or backbone or wire of fixed length is implanted in a joint, then an articular layer or jacket is advanced over the core. For one non-limiting example, a core or backbone or wire is cut to size for a joint and is implanted in a joint, then an articular layer or jacket is advanced over the core. In various embodiments the core could have a feature such as a ball or hook at one or both ends (proximal and distal) so that when the articular layer is advanced over the proximal end of the core the articular lay can recover and butt up against a distal feature or stop. In an embodiment with a proximal feature such as a ball or cap, the articular layer is then trapped or held in position between the features and won't slide off the core. In one embodiment the articular layer can be implanted with no backbone or core.

In one embodiment the articular layer is made of a shape memory material, as described above. In certain embodiments of the orthopedic device 100a, the body of the orthopedic device 100a consists only of an articular layer which has shape-memory properties. In other embodiments, as is described below, additional structures within the articular layer may also have shape memory characteristics. In certain embodiments, the articular layer 105 materials include but are not limited to Silicone, Teflon, Ultra High Molecular Weight Polyurethane or and any implantable grade material. In certain embodiments, the articular layer 105 can be compliant and or compressible or of a non-compressible construction. In certain embodiments, the articular layer 105 can for instance have a variety of durometers (material hardness, such as roughly in the range of 30-90 Shore A). In certain embodiments, the articular layer 105 could also be infused with air bubbles becoming much like a sponge. In certain embodiments, the articular layer 105 can be provided in a number of shapes and be continuous or of interrupted/individual sections. In certain embodiments, the articular layer 105 may contain a material or a drug to inhibit inflammation, joint deterioration etc, or a material or drug to encourage tissue regeneration or device encapsulation. In certain embodiments the articular layer 105 comprises a cartilage replacement material or comprises a natural or synthetic cartilage.

In certain embodiments the articular layer 105 is coated with a drug such as a long lasting steroid. In certain embodiments the articular layer is provided with wells, pockets, porous materials, bubbles or capsules for drug delivery. In one embodiment the articular layer 105 is coated with a secondary surface such as another polymer of a different material property or an antifriction high wear material such as Parylene or other similar materials which are known to the art as providing for a low friction surface.

In one embodiment an orthopedic device 100a can comprise a coating (not illustrated) or covering. The coating or covering could be applied to a core, articular layer, or other surface of the orthopedic device. In one embodiment the coating could be assembled at the time of treatment. In one embodiment, a coating could be a biological covering, such as tissue from the patient in one non-limiting example. Tissue harvested directly from the patient could be harvested using a laparoscope then affixed to the core, articular layer, preshaped ring or backbone and secured to the orthopedic device. The device could then be loaded into a delivery cannula and inserted and ejected (deployed) in the same fashion (method) as the delivery system employed and described herein.

In another embodiment, an orthopedic device is covered with a material, biological agent, or other coating that expands with contact to fluids as may be found in the joint itself. This allows for the insertion of a device of a diameter that is smaller than the fully expanded finished diameter. In one embodiment a coating can be porous. In one embodiment a coating can elute media such as a drug. In one embodiment a coating on the backbone or the articular layer could be hydrophilic in that it could transition from one configuration or diameter (small for insertion) to a larger configuration or diameter when contacting either the body fluid or some fluid provided from an outside source, such as saline. In one embodiment the material, when expanded, can form a casing (or covering) that is spongy or harder or less compliant. This material could also be drug loaded. The casing could form a scaffold for tissue in growth and could be used in joints with unique wear characteristics but not limited to these joints. In one embodiment this concept could also be used in applications where it is of benefit to deliver a filling element, such as an orthopedic filling agent, percutaneously.

In one embodiment the composition of the expanding (swelling) covering could be a composite of a matrix of some polymer combined with a biological material i.e. tissue, cartilage, collagen etc. Note the tissues used in some of these concepts could be cartilage, ligaments, collagen, muscle, etc. In one embodiment, the scaffold could be a polymer-based material. In various embodiments, the casing or covering of the orthopedic device is configured to swell from the small insertion dimension or diameter after implantation to a larger finished dimension or diameter.

In one embodiment an orthopedic device 100a can be comprised of a material or reservoir being drug loaded and dissolvable through features provided in a jacketing or coating material, such as through micro holes, pores, or some other feature. In one embodiment, the orthopedic device could be a drug-loaded element that would slowly dissolve emitting a drug of some sort through a casing that is a spongy and porous. This would leave behind the casing after the ring has dissolved. The benefit could be two fold. First, timed drug delivery could be configured for more controllable dosing. Second, the casing would maintain the space filling or cushioning feature desired and/or allow for tissue organization or in-growth.

In certain embodiments, the articular layer 105 is radiopaque, providing for visibility of the device when implanted as viewed by X-Ray and/or other Fluoroscopic equipment. In one embodiment the articular layer 105 radiopacity is provided by radiopaque markers (not shown here) or by loading the articular layer 105 with platinum, gold or other biocompatible metal.

In various embodiments, any of the features of the articular layer or coatings can be combined on one or more surfaces of the orthopedic device. In one embodiment, an articular layer or coating can provide for tissue ingrowth or fusion with bone, cartilage, or other tissue while another surface provides a low-friction surface to another side of the joint. Any combinations are possible.

As described above, in various embodiments the orthopedic device can be an arcuate, rectilinear or non-straightened configuration once it is implanted in a joint. Some non-limiting examples of arcuate configurations include an open ring (also called an open hoop or an open loop) such as is shown in the embodiment in FIG. 1B, and a nautilus-style spiral as is shown in the embodiment in FIG. 1C. Referring to FIG. 1B, the open hoop arcuate configuration embodiment of the orthopedic device 100b has a proximal end 110b and a distal end 120b in relation to insertion into the body of a patient, such as into a joint. In certain embodiments orthopedic device 100b has many similar attributes and characteristics of orthopedic device 100a, such as shape memory and/or an articular surface 105. In certain embodiments, orthopedic device 100b is an arcuate configuration of orthopedic device 100a. In certain embodiments the orthopedic device of 100a is biased to the configuration as shown for orthopedic device of 100b. The bias may be a preferred configuration for a flexible, pliable, bendable device. In certain embodiments the orthopedic device of 100a can change to the configuration as shown for orthopedic device of 100b by a change in ambient or implantation site temperature or the introduction of an activating medium or material. In certain embodiments, the orthopedic device is reversibly configurable between various shapes or geometries.

In one embodiment the orthopedic device, such as orthopedic device 100b, floats inside the joint to better conform to the natural movement of the bones through the range of motion of the joint. In one embodiment the “open ring,” “hoop” or “coil” configuration or any “open” embodiment including open polygons of an orthopedic device is designed to offer a mechanical advantage over that of fixed type prosthesis as in a total joint replacement as described above in the Background section. The design allows for the distribution of the loading, shearing and/or compressive forces seen by the articulation and or loading of the joint. As open embodiments are not closed, they are not fixed in place (e.g. attached to either end of bones in a joint) and in effect “float” between the ends of the bones in a joint. Thus, in certain open embodiments of the orthopedic device that are flexible, such as orthopedic device 100b, the open configuration offers little to no resistance to shape change and can spring open or closed as force is applied to the device or to the joint, but still maintain the purpose of providing a bearing, cushion, slideable, or articulate surface. In some embodiments, the gap (distance between proximal and distal ends of the device) could be extended to the entire length of the orthopedic device such as when a device is completely straightened. However, embodiments of functional operating ranges allow varying degrees of flexion and gap widening to support loads and articulation in the joint. In one embodiment, the functional flexion in an open orthopedic device allows for a change in the gap between the open ends of the orthopedic device in situ to flex in a range from roughly (or approximately) 0.5 to 6 times the distance between the gap when the orthopedic device is in its natural state in situ. In one embodiment, the flex range is roughly between 2× to 6× (2 times to 6 times) the natural gap distance, and in another embodiment the flex range is roughly 3×-5×, and in another embodiment the flex range is roughly 4×. In one embodiment the functional gap can be as wide as a first dimension, diameter, or width of the over all orthopedic device. As there is little to no resistance to the shape change the orthopedic device 100b in turn allows for the distribution of the forces and/or shear as well as resulting wear along the device more equally. In various arcuate configurations, such as a open circle or continuous spiral, embodiments of the orthopedic device are not closed like a complete ring or closed circular shape would be, resulting in increased dissipation of loading and compression though at least two deformations in the orthopedic device. First, an open ring allows for dynamic loading response as force that is applied to the joint is partially dissipated by the force necessary to radially-outwardly deform the open ring or spiral into a larger radius profile. In one embodiment the operating range of radial deformation of an arcuate orthopedic device is in the range of 0 to 50% of the orthopedic device profile diameter within the joint. Second, as discussed above, the compression of the articular layer resulting in cross-sectional deformation into a flatter shape also dissipates force or pressure in the joint.

In one embodiment the orthopedic device 100b is sized to snugly fit into the joint capsule itself. This fit maintains the orthopedic device 100b center with respect to the axis of the bones of the joint, such as in a finger or a knuckle in one non-limiting example.

In various embodiments the orthopedic device 100b comprises ends which are biased or bent slightly towards or away from its center (see e.g., FIGS. 5B-5E). In one embodiment the orthopedic device, or coil, is out of plane on one or both ends, providing a secondary shock absorbing component to the orthopedic device as the bones in the joint are compressed axially. In one embodiment the orthopedic device 100b is substantially flat, or planar.

One example of a nautilus-style spiral arcuate configuration is the embodiment of an orthopedic device 100c as shown in FIG. 1C. The orthopedic device 100c has a proximal end 110c and a distal end 120c in relation to insertion into the body of a patient, such as into a joint. In certain embodiments orthopedic device 100c has many similar attributes and characteristics of orthopedic device 100a and/or 100b, such as shape memory and/or an articular surface 105. In certain embodiments, orthopedic device 100b is an arcuate configuration of orthopedic device 100a. In certain embodiments the orthopedic device of 100a may be altered in to a configuration as shown for orthopedic device of 100c. The bias may be a preferred configuration for a flexible, pliable, bendable device. In certain embodiments the orthopedic device of 100a when unconstrained can change to the configuration as shown for orthopedic device of 100c, or by a change in ambient or implantation site temperature or the introduction of an activating medium or material. In certain embodiments, the orthopedic device is reversibly configurable between various shapes or geometries.

The orthopedic device 100c floats inside the joint to better conform to the natural movement of the bones through the range of motion of the joint. The nautilus-style spiral arcuate configuration also offers the advantages outlined by the open hoop arcuate configuration, or hoop configuration, but provides a larger bearing surface to the joint. With the extended length of the spiral configuration, the orthopedic device 100c is configured to provide more of an articulate surface, resulting in decreased pressure on the bones by dissipating forces over a larger surface area. The cross sectional diameter multiplied by the number of winds in a spiral shape roughly equals the surface area coverage of the articular surface in conformation with the bones of the joint. For example, a small cross sectional diameter of a spiral configuration allows for a plurality of windings in the spiral. This plurality of spiral windings can then adjust to the general surface area of either bone as the joint articulates.

As described thus far, certain descriptions of embodiments of orthopedic devices have focused on the outside of the device. However, the inside of the devices can have additional structure. For example, in FIG. 2 an orthopedic device 200 according to one embodiment of the present invention comprises an elongate core 240 and an articular layer 230 surrounding at least a portion of the core 240. Referring back to FIGS. 1A-1C, various embodiments of orthopedic devices 100a, 100b and/or 100c can either have an elongate core or lack an elongate core. In other embodiments of orthopedic devices 100a, 100b and/or 100c can either have an articular layer or lack an articular layer. In other words, the orthopedic device may consist of an elongate core, an articular layer, or both. In various embodiments directed to use in knuckles, the cross-sectional diameter or thickness of a core can range from roughly 0.001 to 0.50 inches (0.025-12.7 mm) with some embodiments in a range of roughly 0.005-0.015 inches (0.13-0.38 mm), and some embodiments in a range of roughly 0.01-0.0125 inches (0.26-0.32 mm). In various embodiments, the cross-sectional outer diameter or overall thickness of an articular layer can range from roughly 0.003 to 0.50 inches (0.076-12.7 mm) with some embodiments in a range of roughly 0.039-0.118 inches (1.0-3.0 mm), and some embodiments in a range of roughly 0.078-0.098 inches (2.0-2.5 mm). In some embodiments a ratio of core cross-sectional diameter (or thickness) to articular layer cross-sectional outer diameter (or thickness) can range from roughly 0-500, with certain preferred ranges of ratios from roughly 2 to 30. Other dimensions with the same, similar or different ratios can be used in other parts of the patient's body.

As illustrated in the embodiment of at least FIG. 2 the orthopedic device 200 includes the elongate core 240 in addition to the articular layer 230. One preferred embodiment of the orthopedic device 200 includes an elongate core 240 and an articular layer 230 wherein one or both the elongate core 240 and the articular layer 230 comprise a shape set memory material. In some embodiments the articular layer 230 can surround or encapsulate the entire elongate core 240. In other embodiments the articular layer 230 surrounds, encapsulates, encloses or covers at least a portion of the core 240. As used herein, “surround,” “encapsulate” and “enclose” include configurations in which a core is not completely surrounded, completely encapsulated or completely enclosed. For example, certain embodiments of an orthopedic device contemplate an articular layer which “surrounds” an elongate core with a continuous or non-continuous helical band, discontinuous tabs, or other intermittent articular layer structure.

In one embodiment the articular layer 230 is similar to any articular layer described herein. Likewise, in various embodiments, any articular layer may have some or all of the features of other articular layer embodiments described herein. In one embodiment, the ratio of the cross-sectional size of the elongate core 240 to the articular layer 230 is in the range of roughly 10:1 to 1:10, with a preferred embodiment in the range of roughly 5:1 to 1:5 and another preferred embodiment with a ratio of roughly 2:1.

In one embodiment the elongate core 240 comprises a shape memory material. For example, the elongate core 240 can comprise a shape memory material can made from a heat set/shaped shape-memory material, such as Nitinol or a shape memory plastic, polymeric, synthetic material. For example, one embodiment of the elongate core 240 comprises a shape memory material including a shape memory polyurethane or polyurethane-urea polymer, as is described above. In one embodiment the elongate core 240 comprises a metal “open” ring such as Nitinol encapsulated by an articular layer 230, or outer blanket, comprising silicone. In one embodiment the elongate core 240 comprises a hardened polymer. In one embodiment the elongate core 240 is configured such that a heat set Nitinol with an arcuate configuration, such as an open ring configuration, a horseshoe configuration, or a spiral configuration, can be straightened for delivery through cooling or plastic deformation, then recovered to its original heat-set shape once released from a delivery system, such as one embodiment using a properly sized hypodermic needle. In one embodiment the elongate core 240 comprises a non-shape memory material which can be bent or deformed.

In certain embodiments, the elongate core 240 is coated or impregnated with a drug such as a long lasting steroid. In one embodiment the elongate core 240 is coated with a secondary surface such as another polymer of a different material property or an antifriction high wear material such as Parylene or other similar materials which are known to the art as providing for a low friction surface.

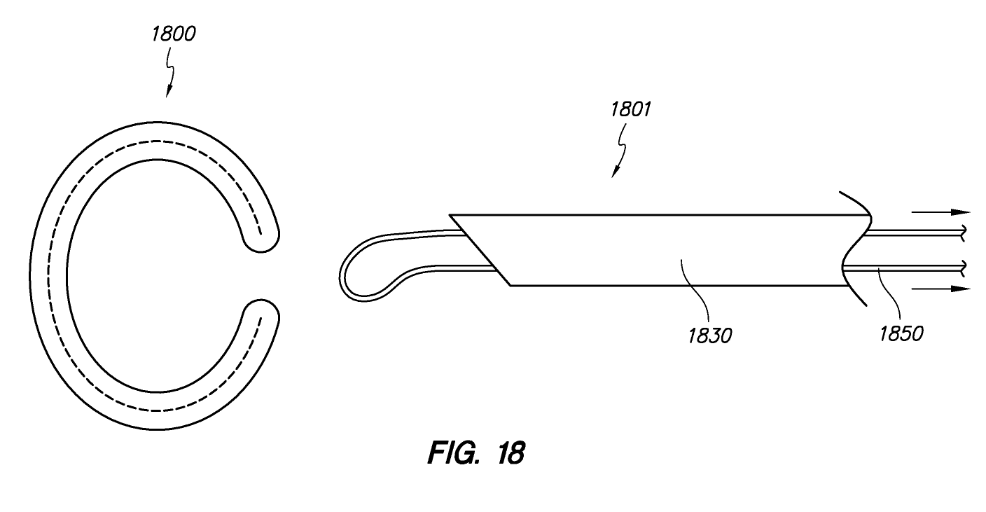

In one embodiment an orthopedic device comprises a removable elongate core and an articular layer. The removable elongate core can be any among the various elongate cores described herein. The orthopedic device would be inserted with an elongate core within the orthopedic device to keep the orthopedic device in a rigid substantially-straight or arcuate shape configuration. When placed in a target site such as a joint in a patient, the removable elongate core could be removed leaving the articular layer in place at the target site. In one embodiment the lumen left in the articular layer by the removal of the elongate core remains hollow allowing for compression, deformation, or cushioning of the joint by the orthopedic device's articular layer (see discussion relating to FIG. 18 below). This lumen, or center, could also be filled with a lumen material such as a liquid, polymer, collagen, or drug etc. The orthopedic device could be provided with a port or a valve at one or both ends to contain the lumen material. In one embodiment the lumen material is a liquid that can be configured, organized or hardened by the application of energy, radio frequency, laser, heat, cold, etc.

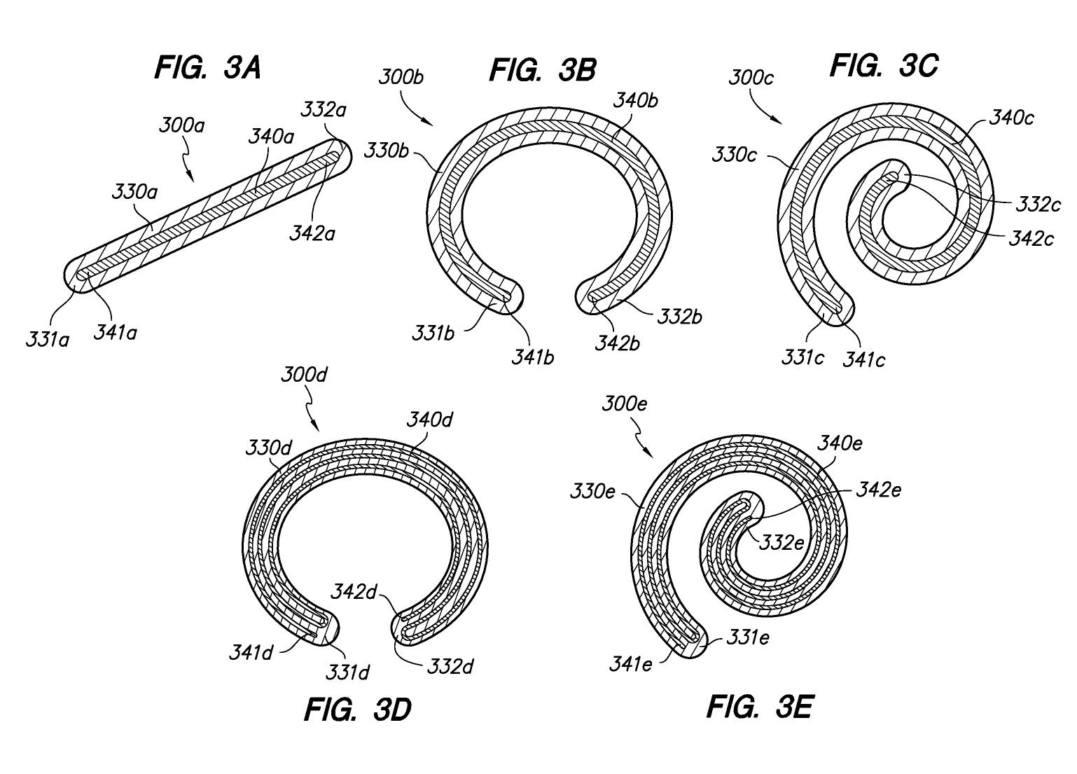

The cross-section of some embodiments of orthopedic devices including an elongate core can have various non-limiting options, as are shown in FIGS. 3A-3E. FIG. 3A is a schematic cross-section of an orthopedic device 300a comprising a substantially straightened configuration. In this embodiment the device comprises an elongate core 340a and an articular layer 330a surrounding at least a portion of the core 340a. The articular layer 330a has a proximal end 331a and a distal end 332a. The elongate core 340a has a proximal end 341a and a distal end 342a342a. In one embodiment the orthopedic device 300a is similar to the orthopedic device 100a described above. FIG. 3B shows a device an elongate core 340b and an articular layer 330b surrounding at least a portion of the core 340b in an open hoop arcuate configuration. The articular layer 330b has a proximal end 331b and a distal end 332b. The elongate core 340b has a proximal end 341b and a distal end 342b. In one embodiment the orthopedic device 300b is similar to the orthopedic device 100b described above. Certain embodiments of a spiral shaped device, such as is shown in FIG. 3C can have a single elongate core. For example, orthopedic device 300c comprises a nautilus-style spiral arcuate configuration, the device comprising an elongate core 340c and an articular layer 330c surrounding at least a portion of the core 340c. The articular layer 330c has a proximal end 331c and a distal end 332c. The elongate core 340c has a proximal end 341c and a distal end 342c. In one embodiment the orthopedic device 300c is similar to the orthopedic device 100c described above.

In some embodiments, the elongate core can wrap around on itself or consist of a number of pieces, such as is shown in FIGS. 3D and 3E. FIG. 3D shows an orthopedic device 300d with an open hoop arcuate configuration. The device 300d comprises one or more elongate cores 340d wrapped, braided or folded along a length of the device and an articular layer 330d surrounding at least a portion of the core(s) 340d. The articular layer 330d has a proximal end 331d and a distal end 332d. The elongate core 340d has a proximal end 341d and a distal end 342d. In one embodiment the orthopedic device 300d is similar to the orthopedic device 100b described above. In the illustrated embodiment in FIG. 3D, the elongate core 340d is a unitary body. In other embodiments, two or more elongate cores 340d are situated in a roughly parallel or co-linear orientation, which can be twisted or braided or interlocked. Other embodiments of the orthopedic device need not be limited to a single elongate core or backbone, but could have a plurality of cores or backbones including a braided configuration, continuous overlaps, etc. FIG. 3E shows an orthopedic device 300e with a nautilus-style spiral arcuate configuration. The device 300e comprises one or more elongate cores 340e wrapped or folded along a length of the device and an articular layer 330e surrounding at least a portion of the core(s) 340e. In the illustrated embodiment in FIG. 3E, the elongate core 340e is a unitary body. In other embodiments, two or more elongate cores 340e are situated in a roughly parallel or co-linear orientation, which can be twisted or braided or interlocked. Other embodiments of the orthopedic device need not be limited to a single elongate core or backbone, but could have a plurality of cores or backbones including a braided configuration, continuous overlaps, etc.

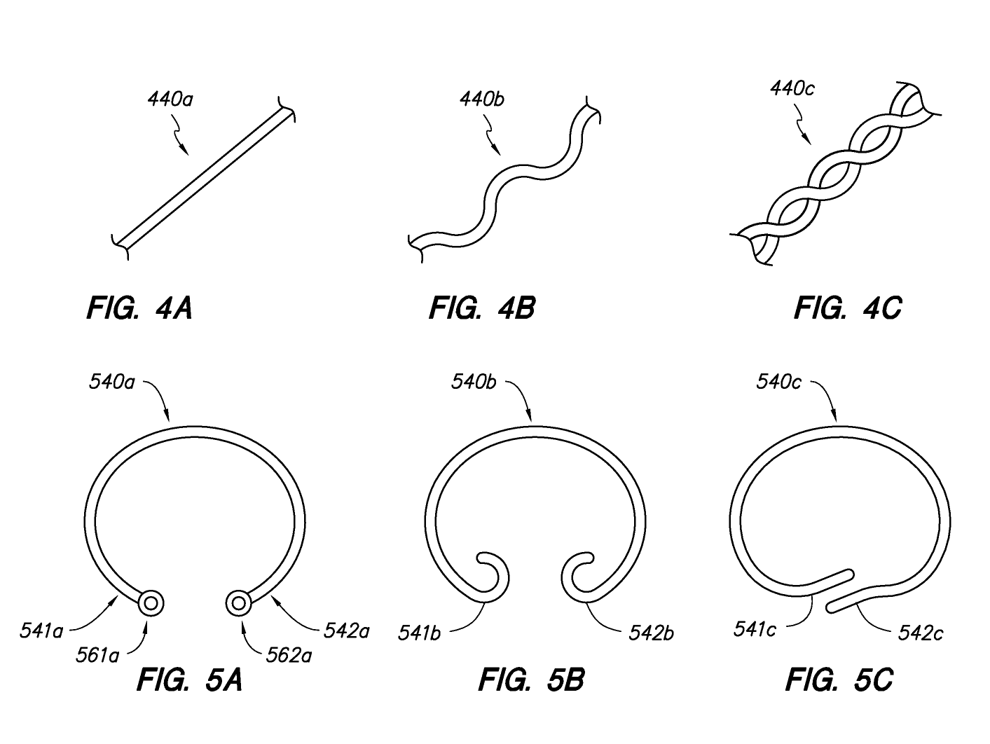

The shape of the elongate core can vary, as is shown in embodiments in FIGS. 4A-4C. FIG. 4A shows an elongate core 440a with one or more substantially linear or straight members. FIG. 4B shows an elongate core 440b with one or more wave, curve or zig-zag members that may be in one or more planes at any angle with respect to one another. FIG. 4C shows an elongate core 440c with one or more members in a braided or weave configuration. Any of these patterns can be used with any of the elongate cores disclosed herein.

Various embodiments of elongate cores can have different features along the length or ends of the core, as is shown in FIGS. 5A-5C. An elongate core 540a with an open hoop arcuate configuration can have one or more end segments, as is shown in FIG. 5A. Such end segments can include proximal end segment 561a and/or distal end segment 562a. In various embodiments, the elongate core or cores 540a can have zero, one, two or more end segments. In one embodiment the end segment 561a or 562a is radiopaque or can be used as a marker for visualization of the ends of the orthopedic device. The elongate core 540a has a proximal end 541a and a distal end 542a. In one embodiment the end segments 561a and 562a are spherical bodies. In another embodiment, the end segments 561a and 562a are loops. In one embodiment the end segments 561a and 562a extend from the same material as the length of the elongate core 540a. In one embodiment the end segments 561a and 562a are separate elements made of the same or different material as the length of the elongate core 540a and which are bonded, fused, welded, glued, or otherwise attached to the proximal end 541a and a distal end 542a, respectively. Although not illustrated, it is contemplated that an elongate core 540a has one or more medial segments anywhere along the length of the elongate core 540a. In various embodiments, elongate core 540a has end segments or medial segments to help improve stability of an articular layer or outer blanket, and need not be flat or planar, but can be biased out of the primary plane of the device at one end or both ends.

One elongate core 540b embodiment includes one or more bends, such as proximal bend 541b and/or distal bend 542b as shown in FIG. 5B. In various embodiments, the bends can also be called hooks. In various embodiments, the bends or hooks can be closed off to form a loop, as with certain embodiments of elongate core 540a. Alternately, elongate core 540c has one or more segments bent in or out of the primary plane of the device as shown in FIG. 5C. In one embodiment proximal segment 541c is bent radially inward from the curvature of the elongate core 540c. In one embodiment distal segment 542c is bent radially outward from the curvature of the elongate core 540c. In other embodiments, proximal segment 541c and/or distal segment 542c are bent radially inward, radially outward, and/or up or down from the primary plane of the elongate core 540c.

FIGS. 5D-5F illustrate non-limiting embodiments of orthopedic devices which may exhibit similar characteristics of other orthopedic devices described above. The embodiments illustrated schematically represent complete orthopedic devices or may represent an elongate core as described herein. FIGS. 5D and 5E illustrate orthopedic devices 570d and 570e, respectively, which have a multi-planar configuration which may be similar to the devices illustrated in FIGS. 1C and 1B or FIG. 3C or 3B. Here, the embodiments of the devices show a characteristic demonstrating that the devices do not have to be constrained in a single plane. FIG. 5F is a schematic side view of an orthopedic device 570f according to one embodiment of the present invention comprising a “W”-shaped generally rectilinear configuration. This embodiment further demonstrates devices that are not limited to arcuate configurations.

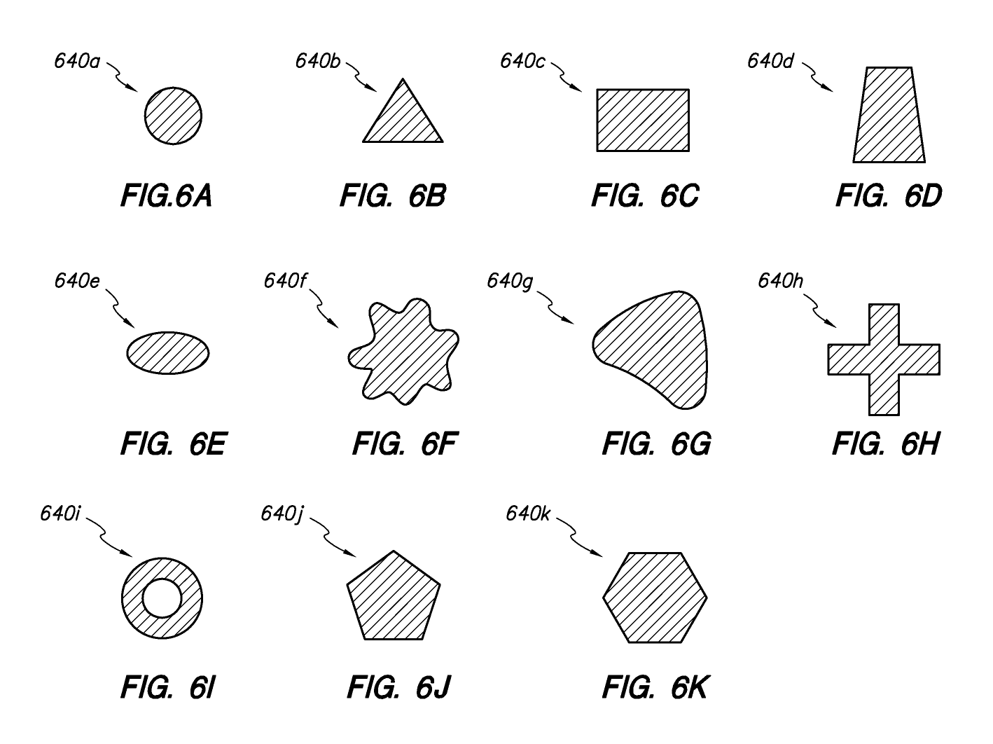

Elongate cores can have any of a variety of cross-sectional structures or profiles. For example, some embodiments of elongate cores cross-sections are shown in FIGS. 6A-6K. The illustrated embodiments are not limiting, but merely examples of various possible cross-sectional profiles of any of the embodiments of elongate cores or orthopedic devices described herein. The illustrated embodiments shows a variety of possible cross-sectional shapes for embodiments of the device or the core of the device, including a square, ellipse, triangular, etc., and wherein the elongate core can be modified by twisting, abrading, pitting and zigzagging, etc.

FIG. 6A illustrates a cross-sectional view of an embodiment of a circular profile elongate core 640a, which can be rotated along a longitudinal axis of the core 640a. In various embodiments the elongate core 640a is at least partially surrounded by an articular layer, wherein the elongate core 640a and/or the articular layer actuate between a straight or slightly curved configuration to a more curved or arcuate configuration. During this change in configuration, elongate core 640a and the articular layer may rotate with respect to each other. In one embodiment the elongate core 640a and the articular layer has some frictional engagement, which may interfere with rotation between the elements, resulting in some level of deformation. Furthermore, in one embodiment both the elongate core 640a and the articular layer will have different material properties which are dependent on stiffness, durometer and other aspects of the respective materials. Depending on the desired orientation of an orthopedic device during delivery to a joint, the orientation of the elongate core 640a and/or the articular layer may be controlled by the configuration of the delivery device being used.

In various embodiments, an elongate core may be configured to limit deformation and/or rotation in various orientations during a change in configuration between straightened and curved profiles. FIG. 6B illustrates a cross-sectional view of an embodiment of a triangular profile elongate core 640b, which can limit rotation of an articular layer along a longitudinal axis of the core 640b. FIG. 6C illustrates a cross-sectional view of an embodiment of a rectangular profile elongate core 640c, which can limit rotation of an articular layer a longitudinal axis of the core 640c. FIG. 6D illustrates a cross-sectional view of an embodiment of a trapezoidal profile elongate core 640d, which can limit rotation of an articular layer along a longitudinal axis of the core 640d. FIG. 6E illustrates a cross-sectional view of an embodiment of an oval or elliptical profile elongate core 640e, which can limit rotation of an articular layer along a longitudinal axis of the core 640e. FIG. 6F illustrates a cross-sectional view of an embodiment of a ridged profile elongate core 640f, which can limit rotation of an articular layer along a longitudinal axis of the core 640f. FIG. 6G illustrates a cross-sectional view of an embodiment of a non-symmetric profile elongate core 640g, which can limit rotation of an articular layer along a longitudinal axis of the core 640g. FIG. 6H illustrates a cross-sectional view of an embodiment of a cross or X-profile elongate core 640h, which can limit rotation of an articular layer along a longitudinal axis of the core 640h. FIG. 6I illustrates a cross-sectional view of an embodiment of a lumen profile elongate core 640i, which can limit rotation of an articular layer along a longitudinal axis of the core 640i. FIG. 6J illustrates a cross-sectional view of an embodiment of a pentagon profile elongate core 640j, which can limit rotation of an articular layer along a longitudinal axis of the core 640j. FIG. 6K illustrates a cross-sectional view of an embodiment of a hexagon profile elongate core 640k, which can limit rotation of an articular layer along a longitudinal axis of the core 640k.

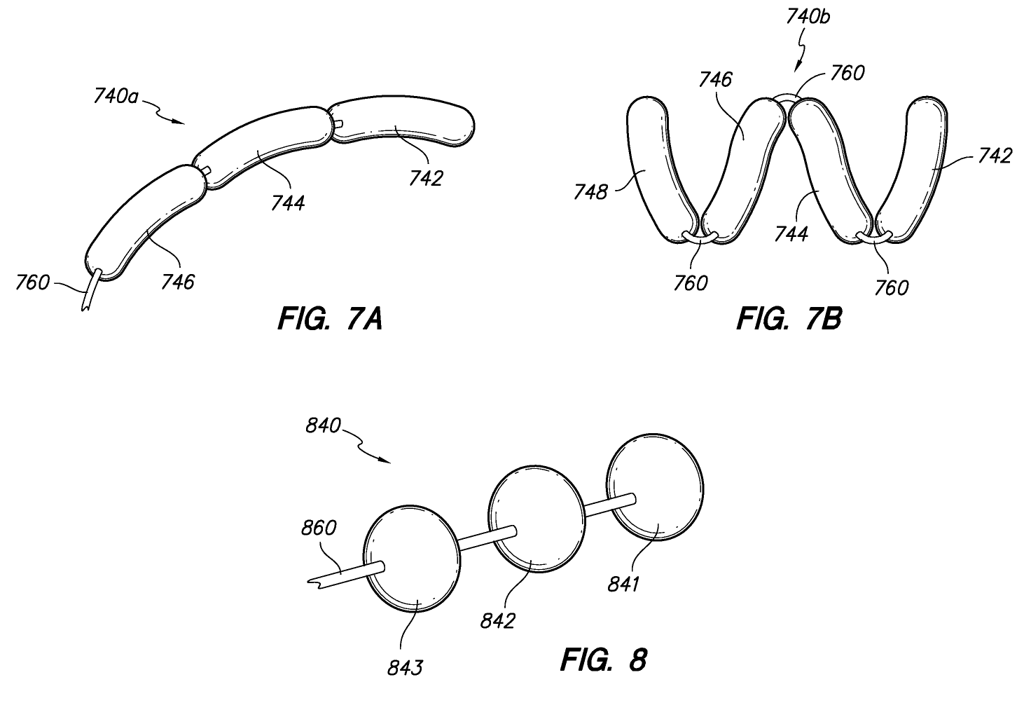

Some embodiments of an elongate core include a plurality of interconnectable discrete elongate members, such as is shown in FIGS. 7-9C. In various embodiments, two or more discrete elongate members may be connected along a single core wire, a series of core wires, or connectors. In one embodiment one or more discrete elongate members can rotate or spin about the connector or core wire. In another embodiment one or more discrete elongate members are affixed to the connectors or core wire in a manner to reduce or prevent rotation of the elongate members with respect to connector or core wire. As illustrated in FIG. 7A one embodiment of an orthopedic device 740a comprising a plurality of interconnectable discrete elongate members has elongate members 742, 744 and 746 which are linked by connector 760. In various embodiments the connector 760 can be a single core member extending between all the discrete elongate members, or it can be any number of discrete connecting members between the elongate members. In one embodiment, an orthopedic device 740b with a plurality of independent or interconnectable discrete elongate members can have a “W”-shaped generally rectilinear configuration. The connectors 760 can be configured to orient the elongate members such as 742, 744, 746 and 748 in any number of orientations or angles. In various embodiments the connectors 760 can have shape memory configurations or biases for particular orientations depending on the doctor's preference or the device selected. The overall shape of an orthopedic device can have any number of configurations: for example, at least a “C”, “O” and “W” shape have been mentioned, but the device and/or articular layer and/or elongate core can be in any shape or configuration. The device is not limited to the “C”-shape or a spiral shape. In one embodiment an orthopedic device is marked with an indicator to indicate orientation of the device. For example, the orthopedic device can be marked with a symbol, text, colors, radiographic markers or inks, or other types of markings that can be sensed visually or otherwise with or without the assistance of sensors or other devices, to indicate a side or feature that should be directed to a specific location. It may be difficult to tell the orientation of an orthopedic device when it deformed to a substantially straightened configuration, thus the marking may provide an indication of the orientation of the device can be helpful for checking proper function or delivery of the orthopedic device.

An elongate core may comprise a plurality of discrete members of one of various shapes and sizes, wherein the discrete members may be interconnected to function as an elongate core or a backbone as set forth herein. Likewise, FIG. 8 shows orthopedic device 840 with interconnected members 841, 842, and 843 which are linked by an extendable connector 860.

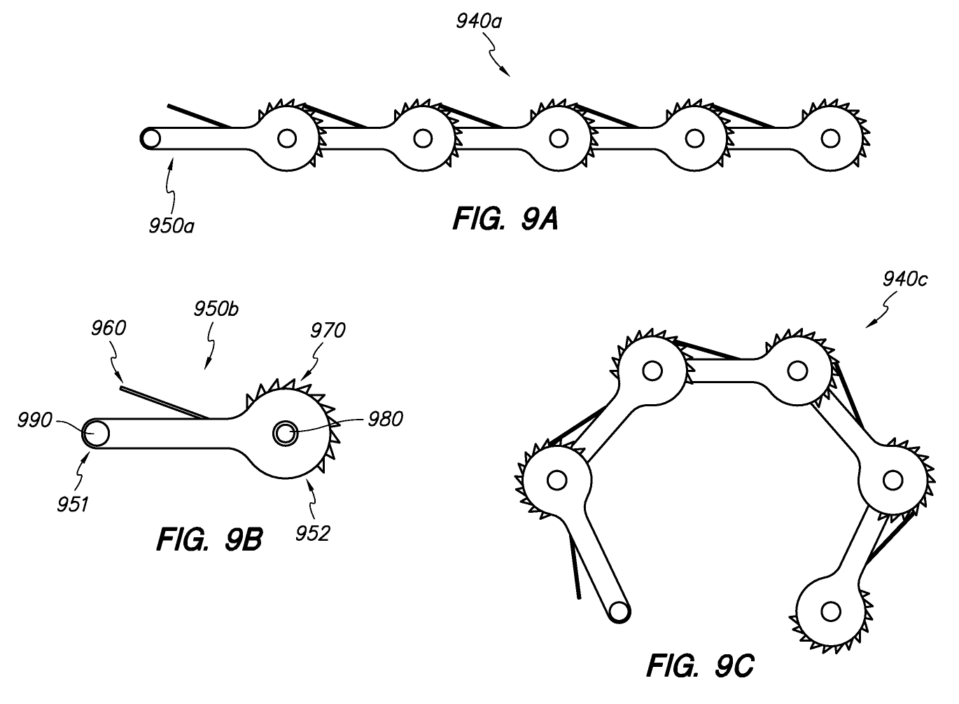

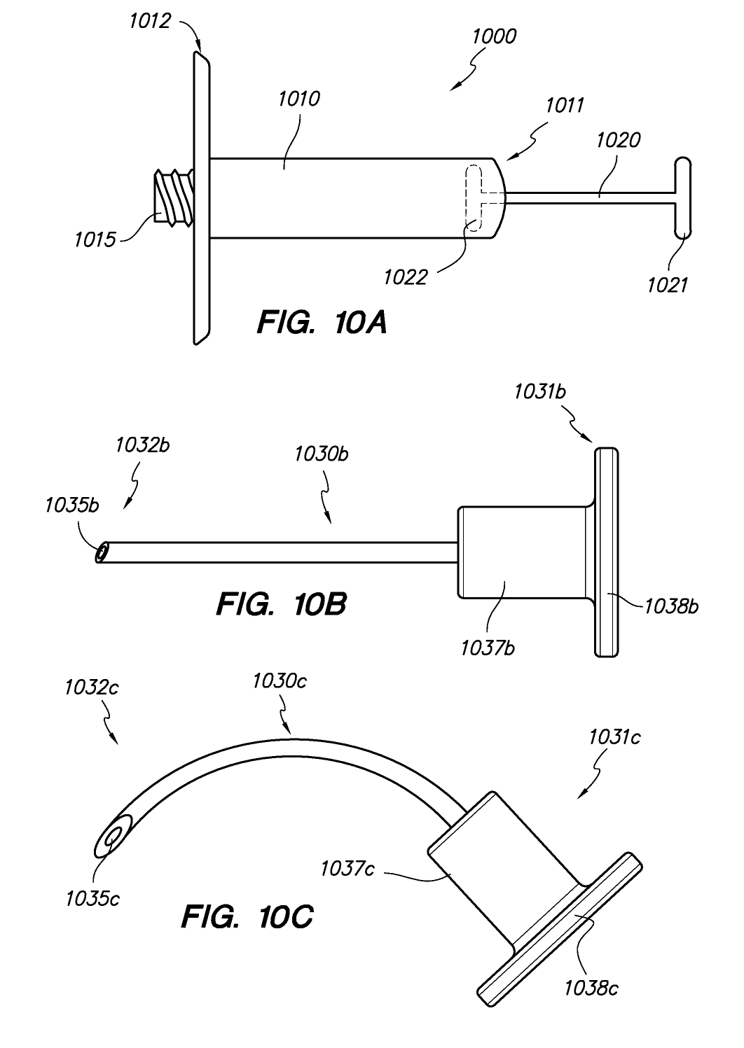

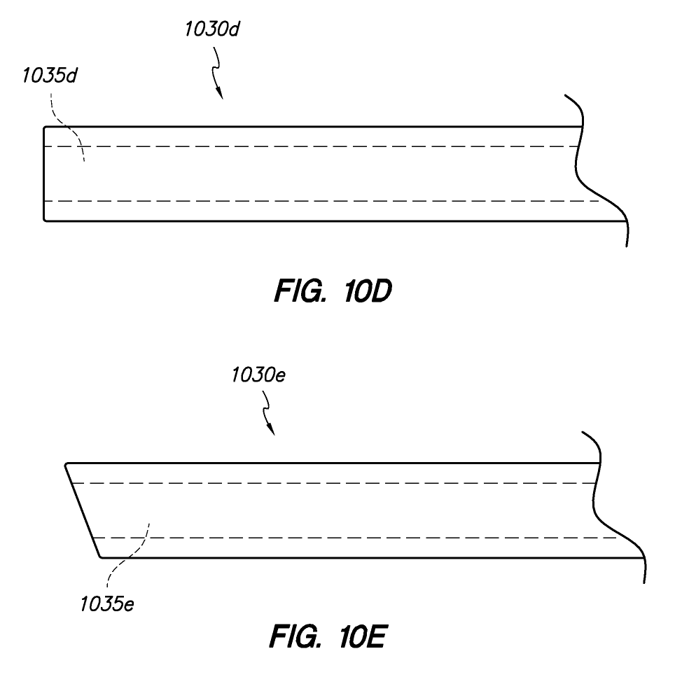

One embodiment of an elongate core 940a with a plurality of interconnectable discrete members, or links 950a, in a substantially straightened configuration is shown in FIG. 9A. Elongate core 940a may be described as a multi-link elongate core, multi-link core, multi-link orthopedic device, or multi-link orthopedic implant. In one embodiment of a multi-link orthopedic device a series of rigid or flexible links are configured to translate the multi-link core from a straight or slightly curved configuration into a curved orientation or configuration. The diameter of curvature of the device could be adjustable by the ratcheting features provided on each link 950a. In one embodiment the links 950a are made of a material that can undergo some level of elastic deformation. In another embodiment, the links 950a are made of a more rigid material. With embodiments of the device, core, or link that are made from a super elastic material such as Nitinol, the implant can be straightened from its curved, deployed or implanted configuration and placed in a needle or cannula. However, a less elastic material such as stainless steel or certain plastics might yield or break if straightened that much. Using a curved delivery system, such as one shown in FIG. 10C below, would allow a more-rigid arcuate implant to be slightly straightened enough for insertion, but not enough to cause yielding.

Looking closer at a link, FIG. 9B shows a side view of one link 950b. In one embodiment link 950b is a link 950a of FIG. 9A. In one embodiment link 950b comprises a first end 951 and a second end 952. Various links 950b are interconnectable between the second end 952 of a first link 950b and the first end 951 of a second link 950b′, and in one embodiment the interconnection is a hinged connection between a first link interface 990 and a second link interface 980. In one embodiment the first link interface 990 is a post and the second link interface 980 is a channel in which the post is captured to allow rotation. In another embodiment, the second link interface 980 is a post and the first link interface 990 is a channel in which the post is captured to allow rotation. In various other embodiments, other link interfaces allowing some rotation including snap fits, connectors, or other similar interfaces may be used. In the illustrated embodiment, the link 950b comprises a ratchet prong 960 and ratchet teeth 970. The ratchet teeth 970 of one link 950b interact with the ratchet prong 960 of a second link 950b′ to allow rotation with respect to links 950b and 950b′ while restricting or limiting rotation in the opposite direction.

Various link embodiments can be configured to an arcuate configuration, as in FIG. 9C showing an elongate core 940c with links according to FIG. 9A in an arcuate open loop configuration. In one embodiment the elongate core 940c is actuated and locked into an arcuate configuration by the ratcheting mechanism as described above. In one embodiment the ratchet locking is configured to be disengageable such that the prong is releasable from the teeth to allow the elongate core 940c to rotate in a straight or less-curved configuration.

2. Methods Apparatus and System for Delivering Implantable Orthopedic Devices

In various embodiments of orthopedic devices described herein, the orthopedic devices are configured to have an arcuate shape in a joint. In certain embodiments, the orthopedic device can be straightened into a substantially straightened or less-curved configuration for implantation with an orthopedic device delivery system. For example, in one embodiment an arcuate orthopedic device can be straightened by cooling or chilling a shape-memory material in the orthopedic device and then inserting the orthopedic device into a tube, cannula, or hypodermic needle of specific design shape and cross section. The pre-loaded hypodermic needle is then attached to a handle through a coupling or interface such as a luer lock standard to the industry or any other attachment means. The physician then straightens the finger by applying force providing for a space or gap to occur in the joint. For example, the force can be provided by using his hands, or a tool, to pull, stretch or spread the desired joint. In one embodiment a sharp tool such as a scalpel or trocar can be used to pierce the joint tissue. In another embodiment, the deliver device needle can pierce the joint tissue. The needle is positioned mid-point between the posterior and anterior surfaces of the joint. The tip of the needle is advanced into the joint, completely within the joint capsule. Once inserted the physician releases the device by advancing it out of the needle using an advancing mechanism, such as a handle and plunger. As used herein, a “plunger” may also be called a push rod, an advance rod, or an advance mechanism. Once deployed the needle and handle can be removed from the joint. If more than one joint, such as a knuckle, is treated the deployed needle can be removed via the luer type connector and a second attached to the same handle, repeating the procedure as needed.

One orthopedic device delivery system 1000 comprising a handle 1010 and a plunger 1020 that is suitable for delivering the orthopedic device implant is shown in FIG. 10A. In various embodiments, the orthopedic device delivery system 1000 can be provided in a number of mechanical configurations. One objective of the orthopedic device delivery system 1000 is to completely advance the orthopedic device out of a channel, cannula, lumen, or needle, with non-limiting examples illustrated in FIGS. 10B and 10C. In various embodiments, the orthopedic device delivery system 1000 is actuated by advancing the orthopedic device by a simple ram type piston or hypodermic needle configuration, or through the use of a lead screw, or through the use of a pneumatic or hydraulic type mechanism. In the illustrated embodiment, the handle 1010 comprises a distal handle region 1012 and a proximal handle region 1011 and the plunger 1020 comprises a distal plunger region 1022 and a proximal plunger region 1021. In one embodiment the distal handle region 1012 comprises a cannula interface 1015, such as a luer connector.

Embodiments of a cannula or needle can be straight or curved, as in FIGS. 10B and 10C respectively. A substantially straight cannula 1030b or needle with a lumen 1035b is suitable for delivering the orthopedic device implant described herein in conjunction with the orthopedic device delivery system 1000 of FIG. 10A. In one embodiment the cannula 1030b comprises a distal cannula region 1032b and a proximal cannula region 1031b. In one embodiment the delivery cannula 1030b can be attached to a handle 1010 in an orthopedic device delivery system such as orthopedic device delivery system 1000 with any of a number of attachment means such as a standard luer type coupler, bayonet, a luer mount, or a thread type means for attachment to the delivery handle 1010. In one embodiment proximal cannula region 1031b comprises a flange 1038b and a luer connector 1037b. The needle or deployment cannula 1030b can be provided in many shapes and cross sections. In one embodiment the cannula 1030b is sized and configured to interface with the orthopedic device in a specific orientation for delivery into a joint. This interface may be a key-slot, or other mechanical interface. In one embodiment the distal cannula region 1032b is provided at its distal end with an insertion feature such as a point, knife edge or blunt atraumatic edge.

Another embodiment of orthopedic device delivery system comprising an arcuate cannula 1030c or curved needle is shown in FIG. 10C. It has a lumen 1035c is suitable for delivering the orthopedic device implant described herein in conjunction with the orthopedic device delivery system 1000 of FIG. 10A. In various embodiments, arcuate cannula 1030c is similar to substantially straight cannula 1030b, except that arcuate cannula 1030c is more curved. In one embodiment the cannula 1030c comprises a distal cannula region 1032c and a proximal cannula region 1031c. In one embodiment the delivery cannula 1030c can be attached to a handle 1010 in an orthopedic device delivery system such as orthopedic device delivery system 1000 with any of a number of attachment means such as a standard luer type coupler, bayonet, a luer mount, or a thread type means for attachment to the delivery handle 1010. In one embodiment proximal cannula region 1031c comprises a flange 1038c and a luer connector 1037c. The needle or deployment cannula 1030c can be provided in many shapes and cross sections. In one embodiment the cannula 1030c is sized and configured to interface with the orthopedic device in a specific orientation for delivery into a joint. This interface may be a key-slot, or other mechanical interface. In one embodiment the distal cannula region 1032c is provided at its distal end with an insertion feature such as a point, knife edge or blunt atraumatic edge.

In some embodiments the process or method of inserting an orthopedic device into a joint is preferably atraumatic. In one embodiment a fluoroscopically placed stab incision is followed by a cannula insertion for orthopedic device delivery. The stab incision would by its nature provide a path for a delivery needle or cannula to follow. The stab incision could or would remove the necessity for the cannula tip to be sharp. For example, In one embodiment a joint such as a knuckle can be physically identified for orthopedic device placement. The device can be fluoroscopically placed or inserted without fluoroscopy. A cannula is inserted into the stab incision and the orthopedic device is delivered through the cannula in the incision to the joint.

Looking more closely at the tip of a needle or cannula, FIGS. 10D and 10E illustrate two potential options. A blunted delivery cannula 1030d with a lumen 1035d is shown in FIG. 10D. In certain embodiments, the blunted delivery cannula 1030d is used in conjunction with a joint piercing tool (not illustrated here) such as a knife, scalpel, spike, trocar, or other sharp instrument for piercing tissue surrounding a joint in order to create an access hole or port through which a cannula can be inserted to provide the orthopedic device access to a joint. An angular tip 1030e with a lumen 1035e is shown in FIG. 10E. In one embodiment the angular tip 1030e is sharp enough to pierce tissue surrounding a joint in order to create an access hole or port through which a cannula can be inserted to provide the orthopedic device access to a joint. In another embodiment, the angular tip 1030e is atraumatic and is used to guide the delivery device in a previously opened incision or natural opening in tissue. Minimally or atraumatic distal cannula regions 1032b, 1032c corresponding to any cannula, such as cannula 1030b-E are intended to be slid through the stab incision, such as made by a scalpel, thereby spreading the tissue which makes up the knuckle capsule as it goes in.

As described above, in various embodiments an elongate core is at least partially surrounded by an articular layer, wherein the elongate core and/or the articular layer actuate between a straight or slightly curved configuration to a more curved or arcuate configuration. During this change in configuration, elongate core and the articular layer may rotate with respect to each other. In one embodiment the elongate core and the articular layer has some frictional engagement, which may interfere with rotation between the elements, resulting in some level of deformation. Furthermore, in one embodiment both the elongate core and the articular layer will have different material properties which are dependent on stiffness, durometer and other aspects of the respective materials. Depending on the desired orientation of an orthopedic device during delivery to a joint, the orientation of the elongate core and/or the articular layer may be controlled by the configuration of the delivery device being used. In various embodiments, the shape, curvature, or tip of the cannula, needle, or lumen can be configured to control the specific orientation of the orthopedic device as it is being implanted. For instance, the point of a needle, trocar, or angle-tipped cannula such as an orthopedic device delivery system with an angular tip 1030e could be used to define the relationship of the orthopedic device and its orientation in a joint.

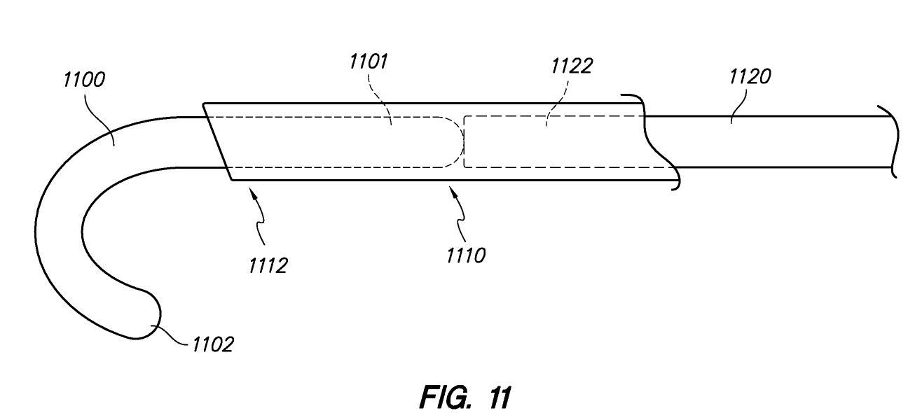

One way of delivering embodiments of the orthopedic device is shown in FIG. 11, where an implantable orthopedic device 1100 is advanced through a cannula 1110 by a plunger 1120. The orthopedic device 1100 comprises a distal end 1102 and a proximal end 1101, and is similar to the embodiments of orthopedic devices described herein. The cannula 1110 has a distal end 1112 that is configured to present the orthopedic device 1100 into the implant delivery site in a joint in the proper orientation. The plunger 1120 has a distal end 1122 which advances the orthopedic device 1100 out of the cannula 1110 and into the joint. In the illustrated embodiment, the distal end 1122 of the plunger 1120 pushes the proximal end 1101 of the orthopedic device 1100. In one embodiment the plunger is sized to match the cross sectional diameter of the proximal end of the device and can also be provided with features to engage the device in a specific fashion. In other embodiments (not illustrated here) the plunger is configured to attach to a distal or medial portion of the orthopedic device to pull or advance the device out of the cannula. In one embodiment an orthopedic device delivery system is configured to deliver a multiplanar orthopedic device from a point corresponding to the distal tip of a cannula into joint. In one embodiment the orthopedic device delivery system is configured to deliver the orthopedic device 1100 in an orientation within a plane (“primary plane”) roughly corresponding to a plane of bony or cartilaginous articulation within a joint that is roughly orthogonal to a longitudinal axis of at least one bone comprising part of the joint. As an orthopedic device is delivered into a joint, such as a knuckle, the tissue surrounding the knuckle including a joint capsule and various ligaments helps maintain the orientation of the orthopedic device in or near the primary plane within the joint by containing the orthopedic device around its outer periphery. In one embodiment an angular tip at the distal end 1112 of the cannula 1110 helps maintain the proper orientation of the orthopedic device 1100 within or near the primary plane and avoiding undesired bias or deformation of the orthopedic device 1100.

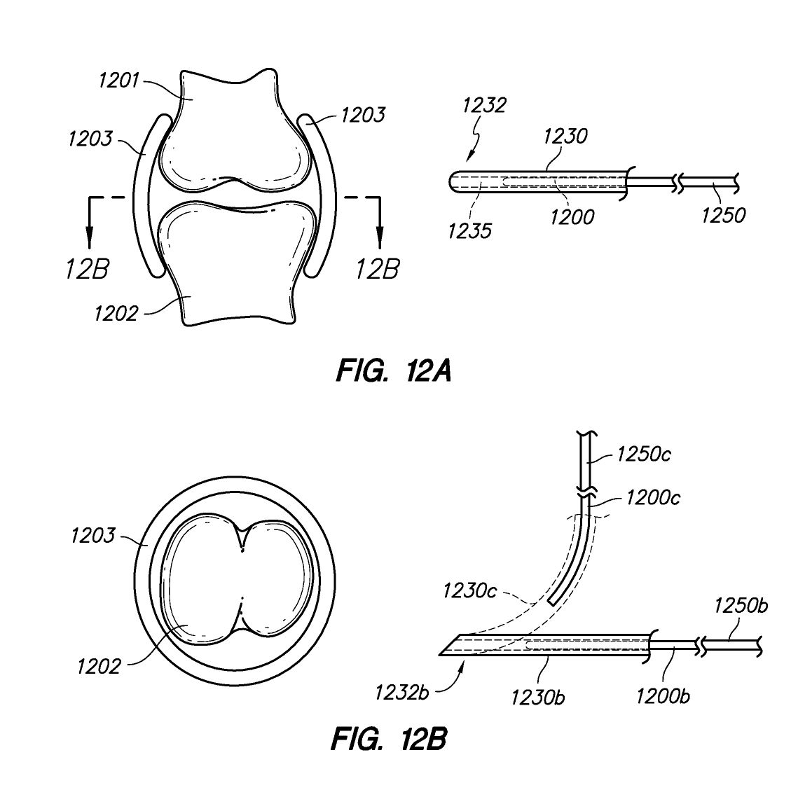

Some of the steps in delivering an orthopedic device 1200 in a joint with an orthopedic device delivery system are illustrated in FIGS. 12A-15B. In these figures a joint comprises a first bone 1201, a second bone 1202, and tissue 1203 surrounding the joint, such as a joint capsule and/or a ligament. The “A” figures illustrate a side view of the joint and the “B” figures illustrate a cross-sectional view orthogonal to the side view in “A.” The primary plane of the orthopedic device roughly corresponds to the plane of the “B” when bones 1201 and 1202 are roughly linear. When the bones 1201 and 1202 actuate with respect to each other, the primary plane may actuate as well to roughly correspond to a plane normal to a point of contact between the bones 1201 and 1202 with the orthopedic device 1200. In one embodiment, the joint is a knuckle. In various embodiments the point of insertion of a cannula into the joint can be anywhere along the periphery of the joint capsule of the knuckle, such as at a side, the top, or the bottom of knuckle, which in one embodiment could correspond to the sides of a finger, the top of the finger (corresponding to the side with a finger nail) or the bottom of the finger (corresponding to the side directed towards the palm). A cannula 1230 with a distal end 1232 and a lumen 1235 is shown in both views. In the illustrated embodiment, the distal end 1232 of the cannula 1230 comprises a feature which helps maintain the proper orientation of the orthopedic device during delivery. As shown, one embodiment of the distal end 1232 feature is an angled tip. In each of FIGS. 12B, 13B, 14B and 15B, two embodiments of a cannula 1230b and 1230c are illustrated. One would be used at a time, but both are illustrated (with cannula 1230b in solid lines and 1230c in dotted lines) to demonstrate that a straight or curved cannula, respectively, can be used to deliver the orthopedic device as described with respect to FIGS. 10B and 10C above. A plunger 1250 advances the orthopedic device 1200 into the joint using any of the advancing mechanisms described herein.

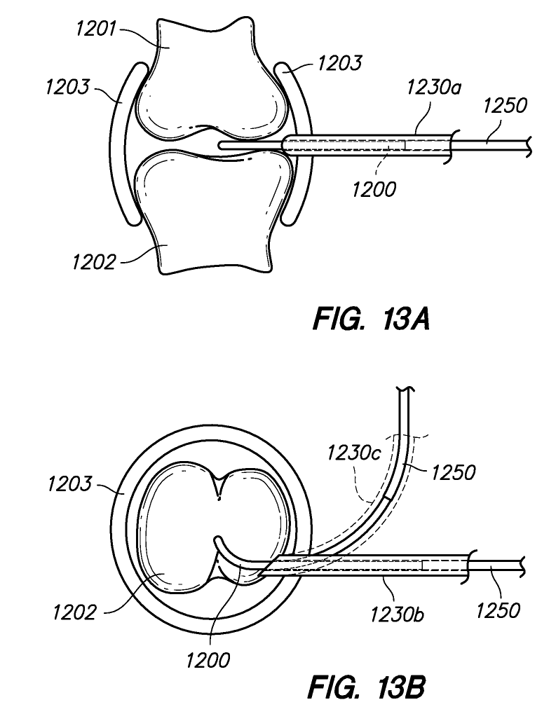

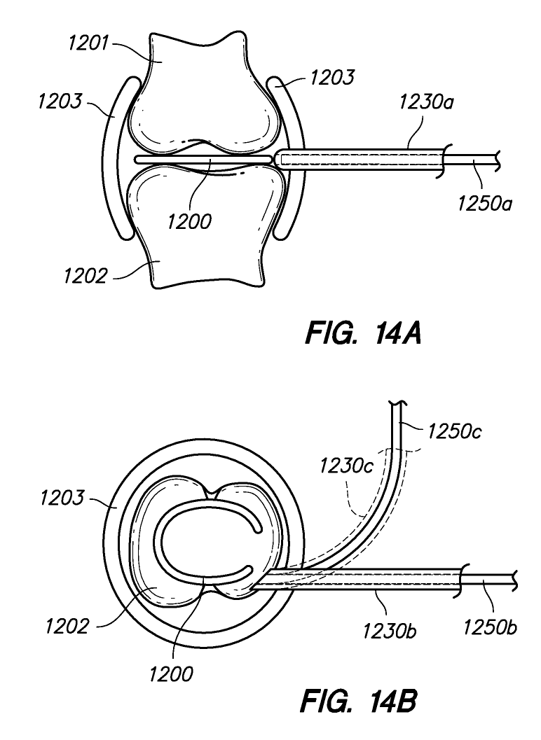

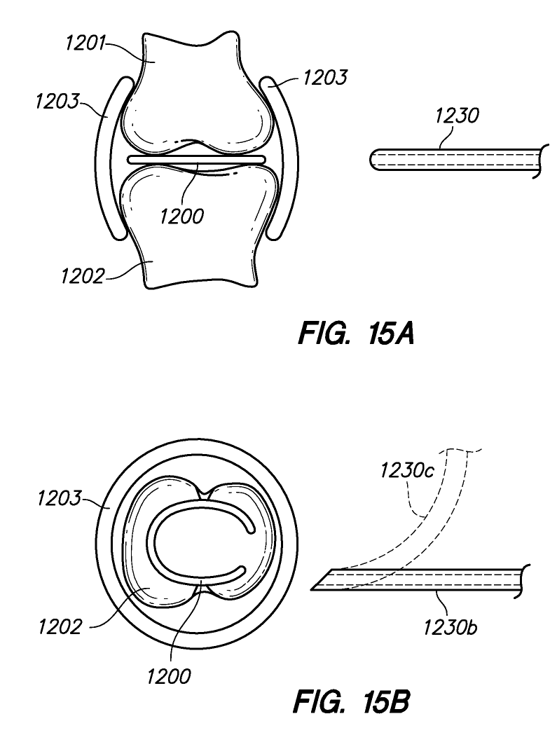

A step showing the device prior to implantation is shown in FIGS. 12A-12B. This illustration shows both a substantially straight cannula 1230b and another embodiment comprising an arcuate cannula 1230c. A step illustrating at least partial insertion of the orthopedic device 1200 into the joint is shown in FIGS. 13A-13B. In one embodiment a tool (not illustrated) is used to pierce the tissue 1203 with a stab incision prior to insertion of the cannula 1230. In another embodiment, the cannula 1230 pierces the tissue 1203. The plunger 1250 advances the orthopedic device 1200 into the joint. Deployment of the device into the joint is shown in FIGS. 14A-14B. The orthopedic device 1200 is shown in an arcuate configuration. The deployment of the orthopedic device 1200 into the joint and removal of the delivery cannula(e) 1230b or 1230c is illustrated in FIGS. 15A-15B.

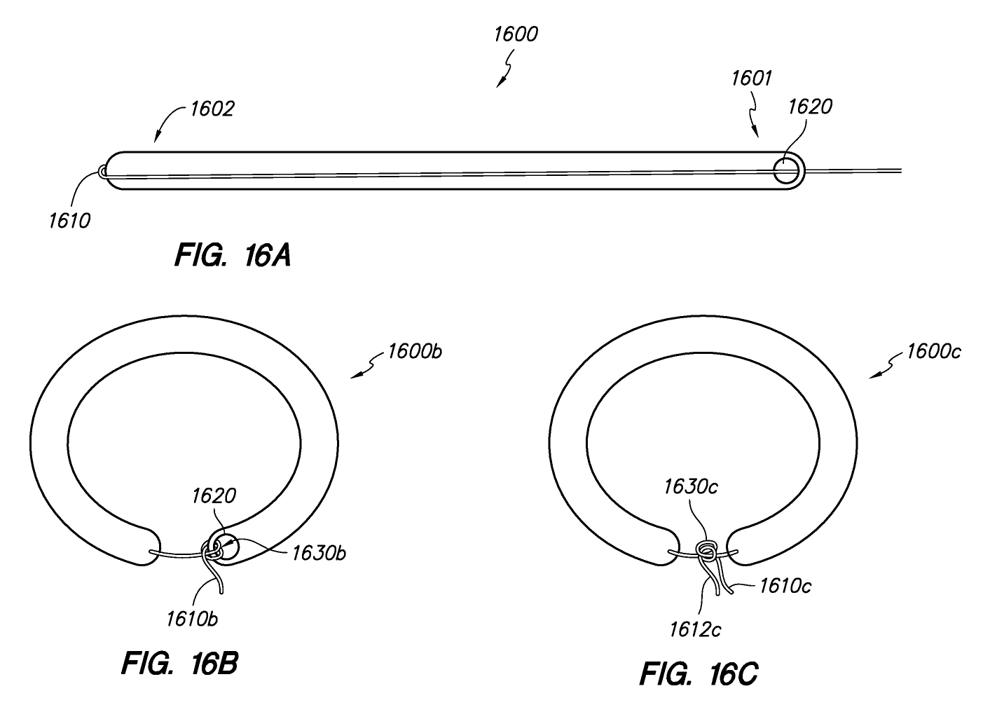

Other embodiments of orthopedic devices can have additional features which can control the extent to which a device is open or closed. For example, one orthopedic device 1600 comprising a tether 1610 and a loop structure 1620 is shown in a substantially straightened configuration in FIG. 16A. In one embodiment, the orthopedic device 1600 exhibits similar characteristics as the previously described devices discussed herein. For example, the straightened configuration of the device 1600 may correspond to a configuration used for device delivery. In a normal state, the device 1600 may be an open ring, arcuate shape, or other configuration or shape when it is not being straightened for delivery or removal. The orthopedic device 1600 comprises a proximal end 1601 and a distal end 1602. The proximal end 1601 comprises the tether 1610 and a distal end 1602 comprises the loop structure 1620. The tether 1610 can be a lanyard, suture, wire, or other structure which in one embodiment is unitary with the orthopedic device 1600. In one embodiment the tether 1610 is unitary with an elongate core in the orthopedic device 1600. The tether 1610 passes through the loop structure 1620. After the orthopedic device 1600 is deployed in a joint it assumes an arcuate configuration as shown in FIG. 16B. In one embodiment the tether 1610b is pulled tight to bring the proximal end 1601 and distal end 1602 of the orthopedic device 1600 toward each other and the tether 1610b is tied into a knot, plug, mechanical fastener or other securing mechanism 1630b to form a substantially closed ring configuration for the orthopedic device 1600b. Depending on the degree of desired openness in the arcuate configuration of the orthopedic device 1600, the tether 1610b can be pulled and locked at different lengths to create a desired hoop or device size. Once the desired size is attained, the securing mechanism 1630b can be locked. The tether 1600b can then be cut proximate to the proximal side of the securing mechanism 1630b and removed from the joint. The tether 1600b can also be used for retrieval of a device that is improperly deployed in the joint or for any reason for removing the device. In one embodiment the tether can be manipulated to reposition an implant, to extract the implant, or it can be cut and pulled out of the joint to pull the implant for retrieval of the device from the joint. In another embodiment an orthopedic device 1600c comprises one or more tethers, such as tethers 1610c and 1612c as shown in FIG. 16C. In one embodiment the tethers 1610c and 1612c are secured to each other with a securing mechanism 1630c such as is described with respect to securing mechanism 1630b. The tethers 1610c and 1612c can then be cut proximate to the proximal side of the securing mechanism 1630c. The tether 1610c and/or 1612c can also be used for repositioning or removal of the tethers or the tethers with the device from the joint, as described with tether 1600b.

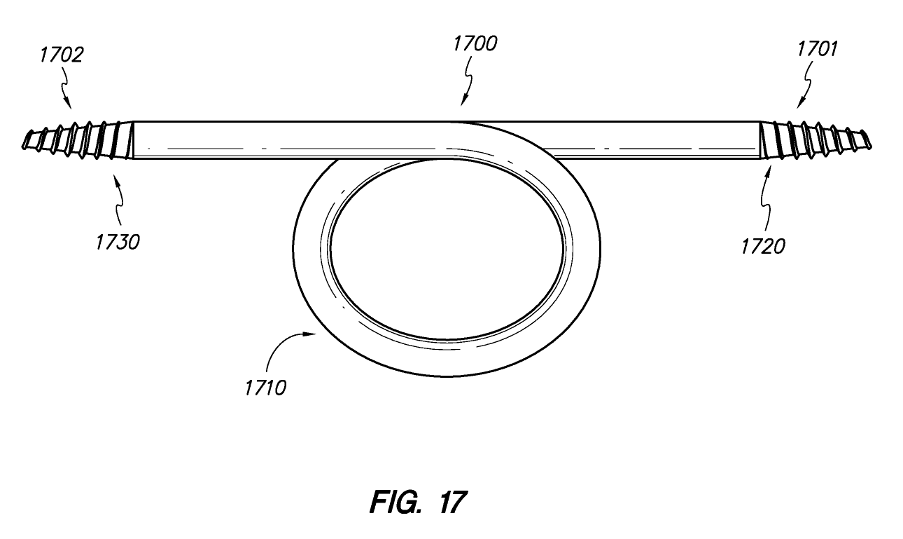

Another embodiment of an orthopedic device 1700 includes a looped arcuate configuration 1710 and at least one anchor, as is shown in FIG. 17. The orthopedic device 1700 has a proximal end 1701 and a distal end 1702. In one embodiment the proximal end 1701 and distal end 1702 are crossing ends on substantially the same axis. In one embodiment the orthopedic device 1700 has a proximal anchor 1720 at the proximal end 1701 and a distal anchor 1730 the distal end 1702. Orthopedic device 1700 has a substantially straight or less-curved configuration (not illustrated) for delivery. Once the orthopedic device 1700 is delivered to the joint, it reverts to its looped arcuate configuration 1710. In various embodiments, the anchors 1720 and 1730 are unitary and formed with an elongate core in the orthopedic device 1700, are unitary and formed with the an articular layer in the orthopedic device 1700, or are formed of separate elements and attached to the orthopedic device 1700. In various embodiments the anchors 1720 and/or 1730 are threaded, tapered, cylindrical, barbed, hooks, ribs, dissolvable, drug eluting and/or non-symmetric. In one embodiment the anchors 1720 and/or 1730 are roughly cylindrical and configured to be releasably attachable with a tool or plunger. In one embodiment the anchors 1720 and/or 1730 are impregnated with a bonding material. In one embodiment the anchors 1720 and/or 1730 are secured in to tissue surrounding or in the joint, such as bone, cartilage, a capsule or ligaments. In one embodiment the anchors 1720 and/or 1730 are bio-absorbable into surrounding tissue.

Retrieval of orthopedic devices is also contemplated. For example, one orthopedic device delivery and retrieval system 1801 can grab an implantable orthopedic device 1800 and pull it through a cannula 1830 using a snare 1850, as is illustrated in FIG. 18. Orthopedic device delivery and retrieval system 1801 is configured to deploy and/or retrieve the implantable orthopedic device 1800. In one embodiment the cannula 1830 is part of a separate retrieval system with a lumen sufficiently sized and configured to recapture and retrieve a deployed orthopedic device 1800. In various embodiments the orthopedic device 1800 has end segments or medial segments along the orthopedic device 1800 articulate layer and/or elongate core, such as is illustrated in FIGS. 5A-5C. In one embodiment the orthopedic device 1800 comprises one or more snare interface points such as end segments 561a and 562a described with respect to FIGS. 5A-5B above. For example, end segments 561a and 562a can be a ball, sphere, bead, hook, loop or other feature which can be ensnared by a tightened snare 1850 to pull the orthopedic device 1800 out of the joint. In one embodiment the snare interface point is radiopaque or has markers for fluoroscopic visualization during the retrieval procedure. In one embodiment the snare 1850 is attached (not illustrated) to a handle or control device proximal to the cannula 1830. For example in one embodiment the snare 1850 is attached to a handle or plunger with can be withdrawn or pulled with respect to the cannula 1830 to tighten the snare 1850 and pull the orthopedic device out of the joint and out of the patient's body.

In one embodiment of an orthopedic device retrieval system 1801 the distal end of the cannula 1830 comprises a hook (not illustrated) which can be used to grab or retrieve an orthopedic device. In one embodiment the cannula hook is actuatable by the doctor by pressing a button to extend or rotate the hook into the joint, which then connects or grabs a part of the orthopedic device for retrieval. In an additional embodiment, the button can be released to pull the hook back into place to lock on to the orthopedic device to be recaptured.

In one embodiment of an orthopedic device retrieval system 1801 only an elongate core is retrieved, leaving the articular layer in the joint in a manner similar to that discussed above regarding FIG. 2.

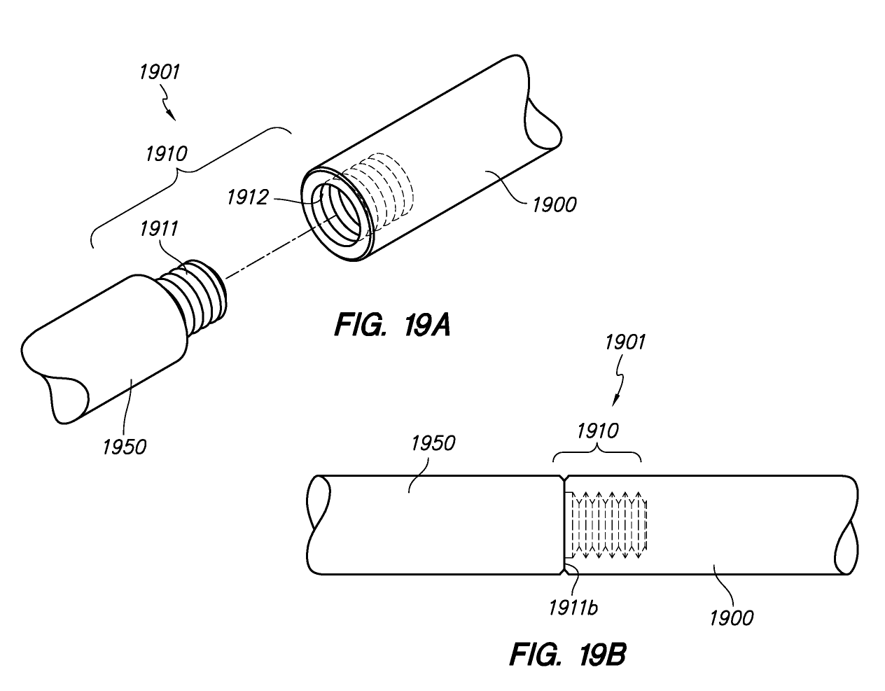

Another orthopedic device retrieval system 1901 can retrieve an implantable orthopedic device 1900 with a plunger 1950 connectable with a device interface 1910, as is shown in FIGS. 19A-19B. One benefit of embodiments of devices connectable with device interfaces is that the connection can allow for final deployment and/or fine-tuning positioning or re-positioning of the orthopedic device once the orthopedic device is out of the cannula of the delivery system. In one embodiment the device interface 1910 is a junction with a male threaded section 1911 on the distal end of the plunger 1950 and a female threaded section 1912 on the proximal end of the orthopedic device 1900. In one non-illustrated embodiment the device interface 1910 is a junction with a female threaded section 1912 on the distal end of the plunger 1950 and a male threaded section 1911 on the proximal end of the orthopedic device 1900. In one embodiment the minor diameter of the threads of the male threaded section 1911 is roughly the same as the outer diameter of the plunger or orthopedic device. In one embodiment the major diameter of the threads of the male threaded section 1911b is less than the outer diameter of the plunger or orthopedic device resulting in a step at 1911b to provide uniform contact with the orthopedic device 1900.

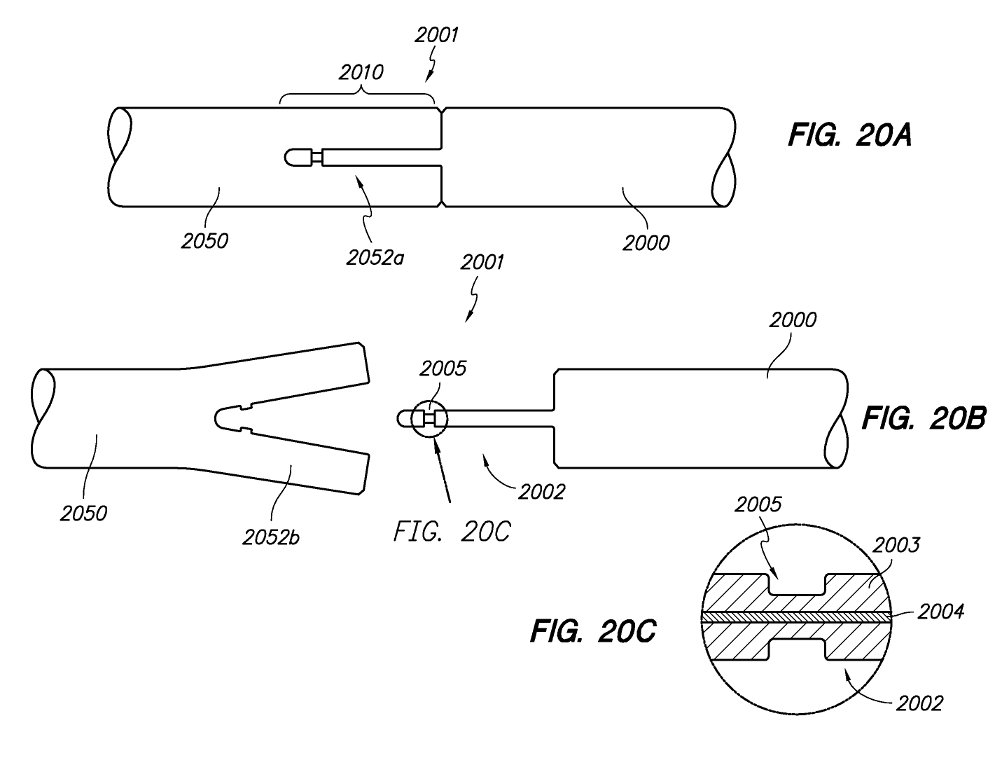

Another orthopedic device retrieval system can remove an implantable orthopedic device 2000 using a plunger 2050 connectable with a device interface 2010, as is shown in FIGS. 20A-20C. In one embodiment the device interface 2010 is a junction with closed jaws 2052a at a distal end of the plunger 2050 and a jaw interface 2002 on the proximal end of the orthopedic device 2000. In one embodiment the jaw interface 2002 comprises a step 2005 for grasping or locking on to the jaw interface 2002. The step 2005 can be a linear, circumferential, or other feature for grasping with the jaws. In various embodiments the jaw interface 2002 comprises a portion of an articular layer 2003, a portion of an elongate core 2004, or, as illustrated in FIG. 20C, both a portion of an articular layer 2003 and a portion of an elongate core 2004 according to various embodiments of elongate cores and articular layers described herein. In one embodiment with the jaw interface 2002 comprising a portion of the elongate core 2004, the elongate core 2004 is exposed at the jaw interface 2002. The closed jaws 2052a can be actuated into open jaws 2052b to release the orthopedic device 2000 into a joint. Conversely, the open jaws 2052b can be actuated into a closed configuration as closed jaws 2052a to recapture the orthopedic device 2000 from the joint. In one embodiment jaws 2052a and 2052b are spring loaded. In alternative embodiments, the device interface 2010 comprises a solenoid, linkage, ring mechanism, push-pin, snap-fit, and ball-detent interface. In one embodiment the device interface 2010 is an electrolyte junction whereby the application of energy, such as electricity, causes the junction to dissolve thereby breaking the junction between the plunger 2050 and the orthopedic device 2000.

In various embodiments an orthopedic device delivery system can be configured to modify the shape or configuration of an orthopedic device between two, three, or more configurations. As shown generally in one embodiment with a pre-loaded needle with FIGS. 10A-10C an orthopedic device can be held in a first configuration (such as a substantially straightened configuration) while stored in a delivery device and actuated to deliver the orthopedic device in to a patient, where the device changes into a second configuration (such an arcuate or rectilinear configuration). In one embodiment, an orthopedic device delivery system can be configured to modify the shape or configuration of an orthopedic device between three configurations: a first configuration in which the orthopedic device is stored in the orthopedic device delivery system, a second intermediate configuration in which the orthopedic device is advanced through a lumen of a cannula in the orthopedic device delivery system in to a patient, and a third configuration in which the deployed orthopedic device is in its proper delivered orientation in the body of the patient. In one embodiment, the first and third configurations may be the same or similar configurations, wherein the first configuration is configured to reduce stress or strain on the orthopedic device while it is being stored by approximating, mimicking, or taking on the identical configuration of the third configuration as deployed in a patient's body to serve its function as an implant. For example, certain embodiments of delivery systems may contain a pre-loaded delivery device wherein an orthopedic device is held in or near its normal, non-straightened configuration, which in various embodiments may include curved, round, or rectilinear configurations that the would be found in the patient's body. The orthopedic delivery device can then be delivered in its proper orientation into the body. Retaining an orthopedic device in or near its normal, non-straightened configuration can reduce strain on the orthopedic device. In one embodiment, an orthopedic device can be removed from the body of patient by moving the orthopedic device from a deployed configuration in situ to a fourth configuration in a retrieval system. In one embodiment, the fourth configuration could be the same or similar to an intermediate or storage system (corresponding to the second intermediate or first storage configurations described above).

Various embodiments of device loaders, loading device, or cassettes can be used to hold orthopedic devices in a first, non-straightened configuration while ensuring proper orientation for delivery of the orthopedic device to the body of the patient. In one embodiment, a loading device provides for minimally invasive delivery in a directed orientation to a joint in a patient. In one embodiment, proper delivery orientation of an orthopedic device is provided by a loading device that orients the orthopedic device such that a proper grip on an orthopedic device delivery system holds the orthopedic devices in a second substantially straightened configuration in a lumen of a needle, catheter or cannula such that plane or orientation such that the orthopedic device exits a lumen into a joint in an orientation or plane that is substantially parallel to a plane between the articulating surfaces of the bone and/or cartilage in a joint. See FIGS. 12A-15B for an illustration of an embodiment of proper orientation of the delivery of an orthopedic device to a joint in a patient, wherein a plane for device delivery is represented in side view in FIGS. 12A, 13A, 14A and 15A corresponding to the plane of the drawing in FIGS. 12B, 13B, 14B and 15B.

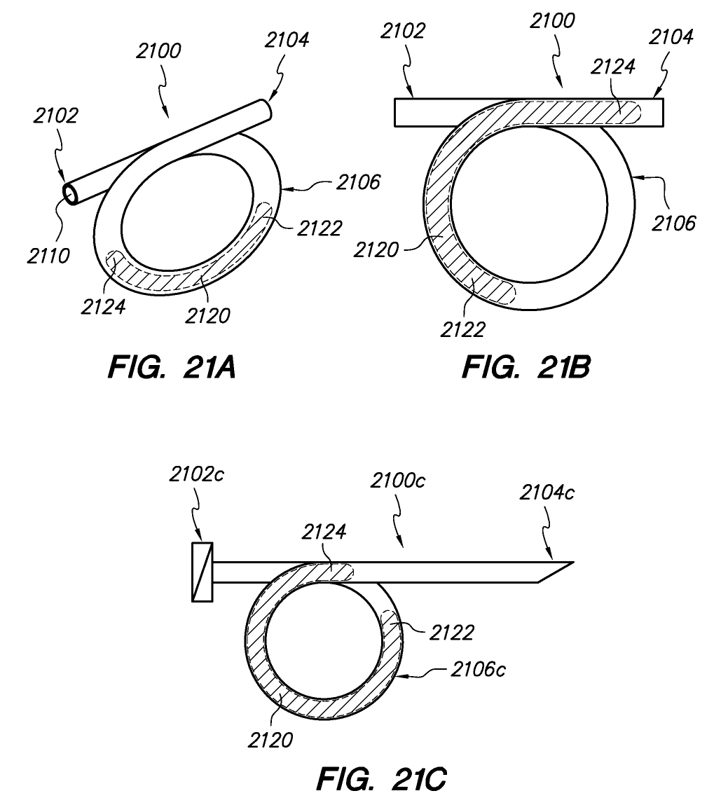

Various embodiments of an orthopedic device delivery system comprise a loading device (also called a device loader or cassette described below in relation at least to FIGS. 21A-28E and 31A-40B) that holds one or more orthopedic devices in a non-straightened configuration until the orthopedic device is substantially straightened for delivery through the lumen of a cannula, catheter or needle into a delivery site in the patient's body. In various embodiments, loading devices can comprise a channel sized to be larger than an outer dimension of the orthopedic device. Although the Figures and discussion may relate to a single orthopedic device, any of the loading device embodiments may be loaded with one or more orthopedic devices that may pre-loaded in the orthopedic device or loadable in an attachable/detachable cassette or loader clip or carrier in which the one or more orthopedic devices can be sequentially deployed using the embodiment's described advancement delivery and/or retrieval mechanism. In various embodiments, the loading device, cassette, loader clip, or carrier can be selectively attached to a delivery system.

FIGS. 21A and 21B illustrate an embodiment of a loading device 2100 comprising a proximal end 2102, a distal end 2104 and a loop 2106 with a channel 2110 (or lumen) extending there through. The loader 2100 is similar to a circular or rounded tube that in one embodiment has the proximal end 2102 and distal ends 2104 continue past one another after they have complete a 360 degree revolution. The channel 2110 is sized to be slightly larger than the outer dimension of the orthopedic device 2120 and is configured to allow the orthopedic device 2120 to be slideably advanced distally into the patient or a needle, or to be slideably retracted proximally. In various embodiments one or both the proximal end 2102 and distal ends 2104 can comprise an attachment interface, such as a connector. In one non-limiting example, the attachment interface can be a luer connector, wherein the proximal end 2102 would connect to a deployment handle or similar structure, and the distal end 2104 would connect to a needle. For example, the proximal end 2102 luer connector could be a female luer configured to attach to a male luer on a proximal device. The distal end 2104 luer connect could be a male luer configured to attach to a female luer on a distal needle. In another embodiment, two or more loaders 2100 could be connected in series in order to deploy two or more orthopedic devices 2120. Advancement of the orthopedic devices 2120 through the one or more loaders 2100 can be accomplished using a flexible plunger (not illustrated here) that is long enough to advance the orthopedic device 2120 out of the one or more loaders 2100. In various embodiments of a loader, a plunger (not illustrated here) can move the orthopedic device from its natural non-straightened configuration to a more straightened or slightly curved configuration in a needle for delivery to a joint.

Another embodiment of an orthopedic device delivery system comprises a loading device 2100c that holds one or more orthopedic devices 2120 in a non-straightened configuration until the orthopedic device 2120 is straightened for delivery through the lumen of a cannula or needle 2104c into the delivery site in the patient's body. FIG. 21C illustrates an embodiment of a loading device 2100c comprising a proximal end 2102c, a distal end needle 2104c and a loop 2106c with a lumen (or channel) extending there through. In one embodiment the loader 2100c has a connector at its proximal end 2102c attachable to a handle, plunger, advancing structure or other loader structure. The needle 2104c is an extension of the distal end of the loader 2100c. In one embodiment the loader 2100c tube is rigid so an operator is able to handle it without flexing or spreading as a plunger, pusher, or advancing mechanism advances the orthopedic device 2120 through the loader 2100c.

In one embodiment of a loader, the loader is made of a material different or dissimilar from the prosthesis or orthopedic device to avoid sticking or jamming or cross linking during a sterilization cycle. In one embodiment, a metal such as stainless steel could be used with a friction reducing layer such as a Teflon liner or coating.

The shape of the lumen extending through the loader 2100c is configured to orient the orthopedic device 2120 is a desired proper orientation for implantation into the body. In one embodiment the lumen can be circular to accommodate a circular cross section orthopedic device. The overall arcuate configuration of the orthopedic device would orient the orthopedic device within the loader with a specific orientation. In other embodiments, the lumen of the loader can have a specific cross section shape or key or feature at one or more points along the loader, or along the entire length of the loader, to orient the orthopedic device in a specific orientation for delivery or retrieval.

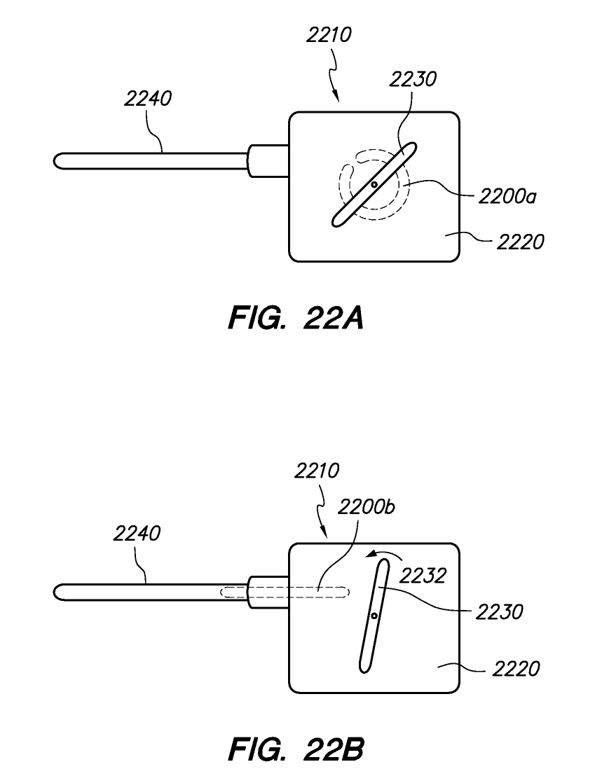

One embodiment of an orthopedic device delivery system 2210 comprises a loading device 2220 (also called a device loader or cassette) that holds one or more orthopedic devices 2200a in a non-straightened configuration until the orthopedic device 2200a is straightened for delivery through the lumen of a cannula 2240 or needle into the delivery site in the patient's body. In one embodiment, as illustrated in FIGS. 22A and 22B, one the orthopedic device delivery system 2210 comprising a loading device 2200 (cassette) that contains and/or stores one or more orthopedic devices 2200a in a curved configuration. The loading device 2220 can be single use, disposable, or re-usable. The loading device 2220 is removably attachable to any previously described delivery or retrieval system, including embodiments with plungers, etc. or with any embodiment described in conjunction with FIGS. 10A-15C. As illustrated, the embodiment of the loading device 2220 is removably attachable to a needle or cannula using any attachment configurations, such as a snap fit, lock, threaded engagement, or form fit. In one embodiment, the loading device 2220 comprises an advancement mechanism, such as by spring loading or manual advancement, such as using a knob 2230 to advance the device. When the knob 2230 is rotated or actuated, the orthopedic device 2200a is loaded into the lumen of the needle 2240 in a specific, proper orientation, and changes configuration to a more straightened form as shown with orthopedic device 2200b. The various embodiments of loading devices can be used with any of the embodiments of the orthopedic devices described herein.

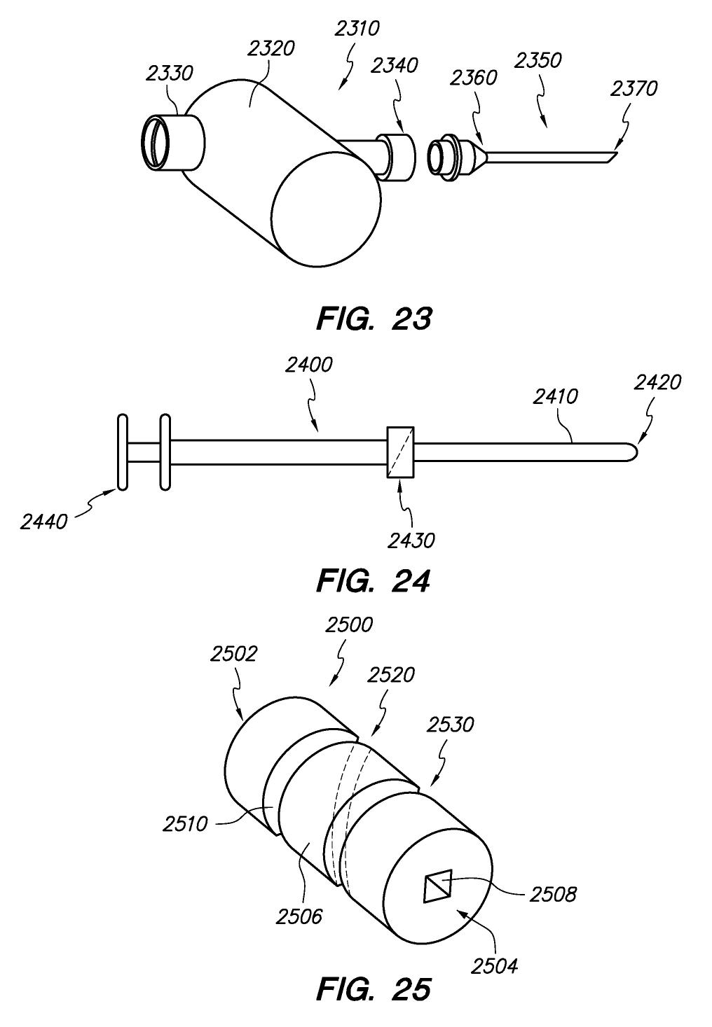

One embodiment of an orthopedic device delivery system 2310 comprises a cassette 2320 with a proximal interface 2330 and a distal interface 2340. The cassette 2320 can hold one or more orthopedic devices in a curved, rounded or rectilinear configuration until the orthopedic device is straightened for delivery through the lumen of a needle 2350 or needle into the delivery site in the patient's body. As illustrated in FIG. 23 one embodiment of an orthopedic device delivery system has a proximal interface 2330 that can mechanically and releasably connect to an orthopedic device advancement mechanism such as a plunger in a handle. A distal interface 2340 can mechanically and releasably connect to any embodiment of needle or cannula described herein. One embodiment of a needle 2350 has a distal end 2370 for insertion into a joint and proximal interface 2360 which connects to the distal interface 2340 of the cassette 2320.

Various embodiments of an orthopedic device advancement mechanism, such as a plunger in a handle, may be employed to move the orthopedic device proximally or distally depending on the interface between the plunger and the orthopedic device. Various interfaces are discussed above. In various embodiments the handle advance is used to move the device into the delivery needle from a cassette. In certain embodiments the plunger itself is flexible and has the same or similar diameter as the orthopedic device. The plunger is also of a specific or fixed length such that it advances the orthopedic device to its exact position within the needle. One embodiment of a plunger 2400 is illustrated in FIG. 24. Plunger 2400 has a proximal end 2440 and a distal end 2410. The proximal end 2440 may be manually or mechanically advanced, and in one embodiment, is similar to the proximal end of a plunger for use in a hypodermic syringe. In one embodiment, the proximal end 2440 and the distal end 2410 are removably attachable in an interface 2430. In another embodiment, the proximal end 2440 and the distal end 2410 are permanently attached. In one embodiment the distal end 2410 is a flexible member configured to fit within a lumen of a needle to slideably advance an orthopedic device through the lumen. In various embodiments the distal end 2410 can be rigid or bendable and may have a distal tip 2420 of the distal end 2410 that is blunt and/or releasably attachable to the orthopedic device.

In one embodiment, a cassette barrel 2500 fits with in the cassette and engages the orthopedic device in such a way as to position the device in the proper orientation for implantation or extraction. FIG. 25 illustrates one embodiment of an interior component of a cassette in an orthopedic device delivery system. In one embodiment the cassette barrel 2500 comprises a first side 2502 and a second side 2504 with a generally or substantially cylindrical surface 2506 in between. In one embodiment a groove 2510 is recessed into the cylindrical surface 2506 to contain and guide the orthopedic device and/or a flexible plunger or a plunger tip 2410 as described above. In one embodiment the plunger travels in the helical groove 2510 shown in the “barrel.” In one embodiment the orthopedic device is loaded into the helical pitch or groove 2510. In one embodiment the proximal end of the orthopedic device matches the entry sight of the handle connector 2330 corresponding to a portion of the groove labeled 2520. The distal end of the orthopedic device is aligned with the delivery needle connection 2340 corresponding to a portion of the groove labeled 2530. The helical pitch of the groove 2510 is cut deep enough to accept the full diameter (cross-section) of the orthopedic device. For instance if the device is a 2 mm device the groove 2510 width and/or depth can be a little larger than 2 mm. In one embodiment, the groove 2510 also accepts the full diameter (cross-section) of at least a portion of the plunger 2400, such as the distal portion 2410, which travels within the groove 2510 to push the proximal end of the orthopedic device distally.

In one embodiment the overall barrel 2500 diameter within the groove 2510 can be larger, smaller or equal to the normal shape or diameter of a rounded (or non-straightened) configuration of the orthopedic device. The groove 2510 can be used to hold the orthopedic device in the cassette 2320 at or near its round (normal) shape. Once the orthopedic device is pushed or pulled from the cassette 2320 into a cannula or needle such as needle 2350, the orthopedic device can assume a straight or slightly curved shape. In one embodiment the helical groove 2510 is configured to hold the orthopedic device in its normal non-straightened shape with the ends of the orthopedic device offset so it can be pushed at its proximal end to advance its distal end.

In one embodiment the groove 2510 has features to aid to aid in the saline flush for lubrication of the orthopedic device, with one example of lubrication being supplied prior to implantation. These features could be micro grooves along the walls of the groove 2510 itself. In one embodiment the system could be flushed with sterile saline prior to orthopedic device delivery through the handle connector. Additionally, the groove 2510 in the barrel 2500 could have one or a plurality of micro grooves along its length allowing for a substance, such as silicone, to be flushed when the saline is injected.

In one embodiment the barrel 2500 is contained by the outer housing 2320. In one embodiment the barrel 2500 is keyed to assure proper orientation within the outer housing 2320. The first side 2502 and/or the second side 2504 may have a key slot 2508 at or near an axis. In one embodiment, the cassette barrel 2500 is contained within the cassette housing with an external handle or knob connected to the key slot 2508 in order to rotate the cassette barrel 2500, thereby advancing or retracting an orthopedic device. In another embodiment, the cassette barrel 2500 is held by the cassette housing with an interlocking key feature on the inside of the cassette housing which locks the key slot 2508 so that the cassette barrel 2500 does not rotate. Note that the illustrated square “key” feature shown in one embodiment at the center of the barrel can be used if the barrel is a separate component to the cassette housing assembly and would need to be oriented in a specific fashion when assembled to the cassette. In another embodiment the cassette 2320 can be comprised of an integrated housing and barrel structure, where the handle/plunger and cassette can be one piece (e.g. permanently attached or formed into a single structure). Various needles can be attached depending on the desired size, indication, and orientation intended for the implant.

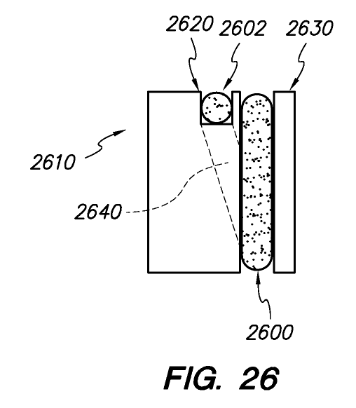

Another view of a barrel 2610, which can be similar to the barrel 2500, is shown in FIG. 26. In one embodiment a groove 2620 can be configured to house or guide an orthopedic device 2600 with a proximal portion 2602. A portion of the groove can be slanted 2640, curved, straight 2630, spiral, helical or some other shape to ensure the orientation of the orthopedic device 2600 is proper for delivery to the patient. In one embodiment a plunger, as described above, can be housed in the groove 2610 for slideably advancing or retracting the orthopedic device 2600. In various embodiments the groove 2610 can be oversized to house the orthopedic device or plunger, can have a square or rounded cross sectional shape or any other configuration, and can direct the orthopedic device 2600 along a path which terminates at a feature which guides (straightens) the orthopedic device 2600 for insertion into the delivery needle.

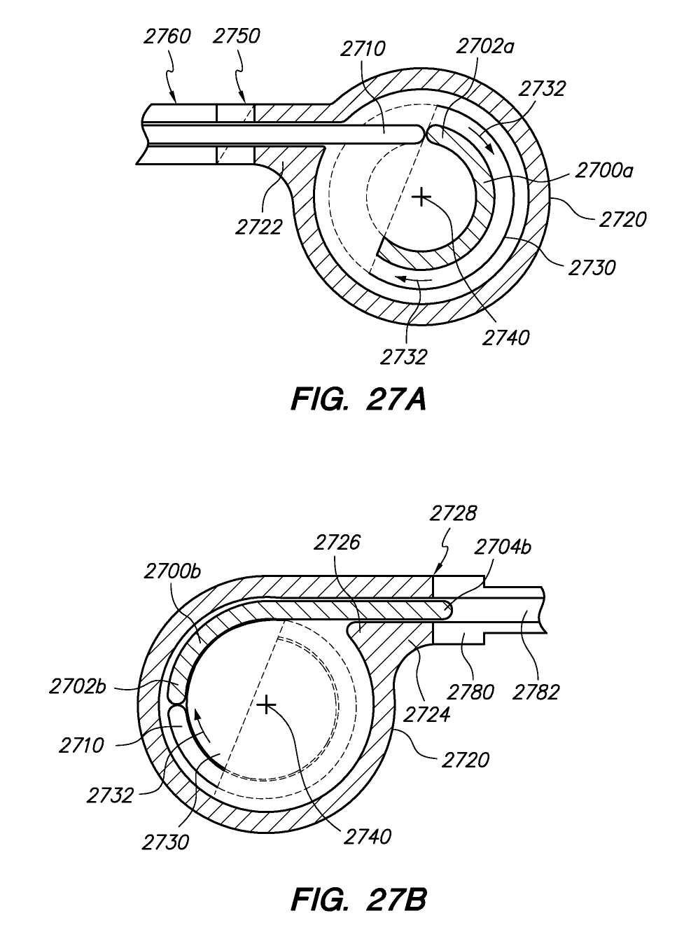

As described above, in one embodiment a flexible plunger can be used to advance or retract an orthopedic device through a cassette and into a straight or straightened configuration for delivery through a needle. FIGS. 27A and 27B show partial cut-away schematic side views of one embodiment of the advancement of an orthopedic device 2700a and 2700b distally from a cassette 2720 into a needle 2780 that is permanently or removably attachable at a connection 2728 near the distal end 2724 of the cassette 2720. A plunger 2710 is advanced through a lumen in a handle or advance mechanism 2670. The advance mechanism 2760 and the cassette 2720 are permanently or removably attachable at a connection 2750 near the proximal end 2722 of the cassette 2720. The plunger 2710 advances into a groove along a barrel 2730 with a center 2740 to advance the proximal end 2702a of the orthopedic device 2700a (which is also shown as proximal end 2702b of the orthopedic device 2700b in FIG. 27B) in a direction 2732. The distal end 2704b of the orthopedic device 2700b advances over an alignment ledge 2726 near the distal end 2724 of the cassette 2720. As the distal end 2704b of the orthopedic device 2700b advances in the lumen 2782 of the needle 2780, the orthopedic device 2700b straightens out into a slight curve or a straight line configuration.

In various embodiments there could be more than one orthopedic device per cassette or multiple cassettes joined together.

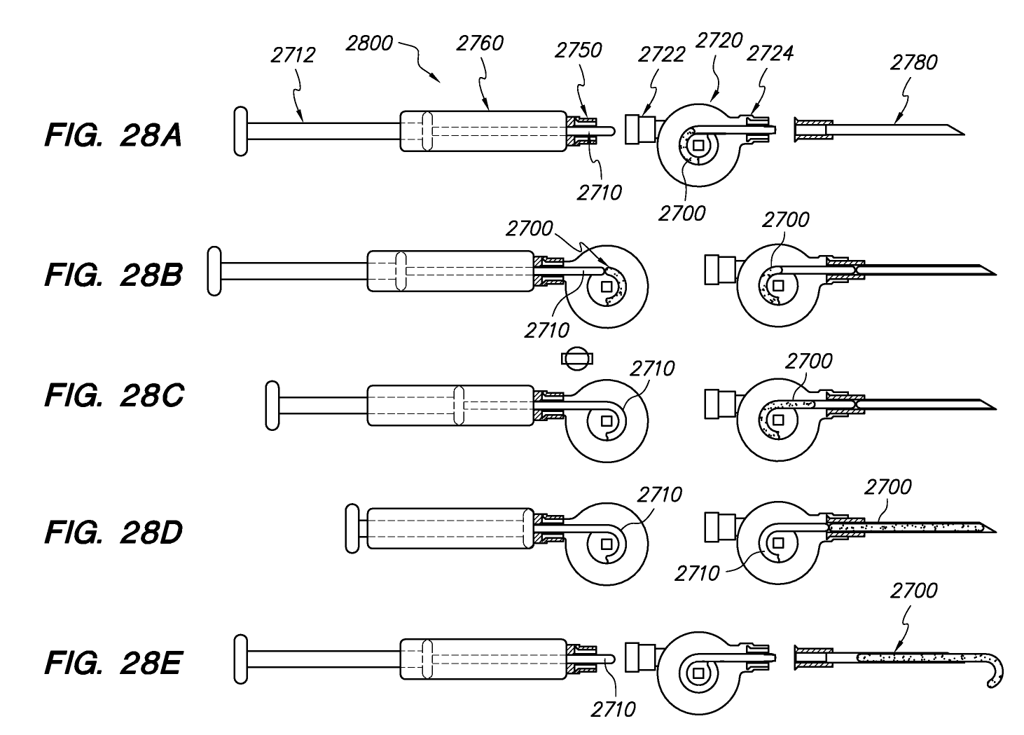

One embodiment of an orthopedic device delivery system 2800 comprising a cassette, barrel and plunger that is similar to embodiments described above is shown in FIGS. 28A-28E. The series of figures helps illustrate the advancement of an orthopedic device 2700 with a plunger advancer 2712 in a plunger body 2760 with a plunger luer connector 2750 and a plunger distal portion 2710. The cassette body 2720 has a proximal plunger luer connector 2722 and a distal delivery needle luer connector 2724 that is removably attachable to a delivery needle 2780. As the plunger advancer 2712 moves distally, the flexible plunger distal portion 2710 advances into the cassette 2720 and bends around to push the orthopedic device 2700 out of the cassette 2720 and into the needle 2780. The distal tip of the plunger distal portion 2710 advances forward pushing the proximal end of the orthopedic device 2700 along a groove provided in a cassette barrel as shown in FIGS. 23-27. The plunger 2712 advances the device 2700 completely into the needle 2780 to a predetermined depth. In one embodiment the device 2700 is detached from the plunger distal portion 2710 (with any of the embodiments of attachment mechanisms described herein, such as with FIG. 19 or 20) and the needle 2780 is withdrawn from the implant delivery site.

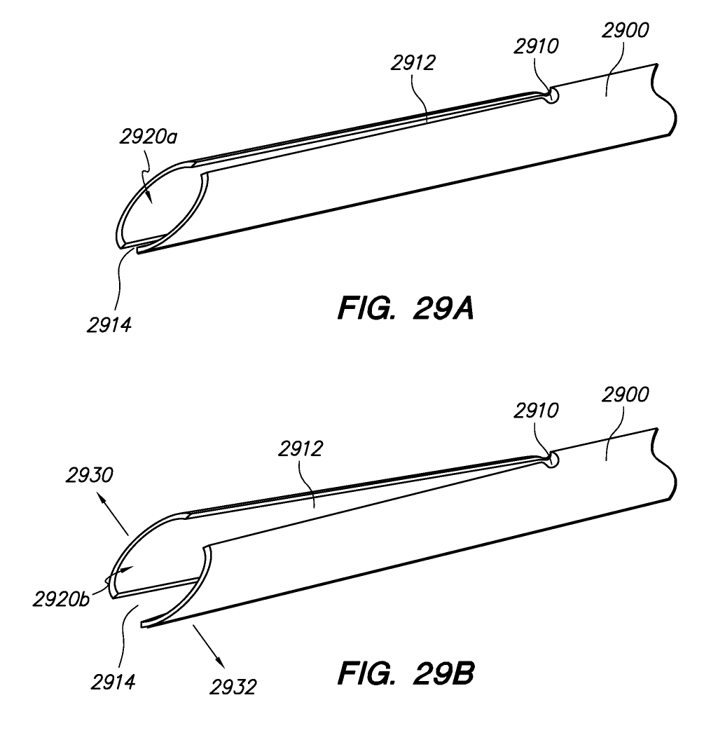

In one embodiment of a slotted needle 2900 that can be used with any of the embodiment of an orthopedic delivery device system described herein the slotted needle 2900 may be used to spread the bones of a joint apart as the delivery device is advanced into the joint. In the embodiment illustrated in FIGS. 29A and 29B the slotted needle 2900 has a stress relief 2910 and at least a first slot 2912. In one embodiment the slotted needle 2900 has a second slot 2914. In one embodiment the lumen or bore of the needle is undersized to the prosthesis or orthopedic device. When the needle is advanced into the joint it has a small diameter. Then when the prosthesis is advanced through the bore it forces the split barrel of the needle outwards. This outward force may be sufficient to influence/spread bone or tissue outwardly from the centerline of the needle. In the illustrated embodiment the needle lumen expands from a first configuration 2920a to a second configuration 2920b, where the sides of the needle at the distal end of the slotted needle 2900 can be moved apart in directions indicated by arrows 2930 and 2932. The distal slotted end of the needle can also be narrowed so that it slips in between the bones. Then as the orthopedic device is advanced distally it urges (pushes) the bones of the joint apart. In one embodiment a radiopaque strip or marker can be provided on one or both edges of the slot for orientation. In various embodiments one or more slots 2912 and/or 2914 can be 0 to 100 mm long, can have a slot width from 0.001 thousandths of an inch to 0.025 thousandths of an inch, can have a slot width that can vary from slot to slot or along the individual slot, can be straight, can be curved, can be spiral, can have a proximal end of the slot that is provided with a pivot to allow for springing apart without cracking.

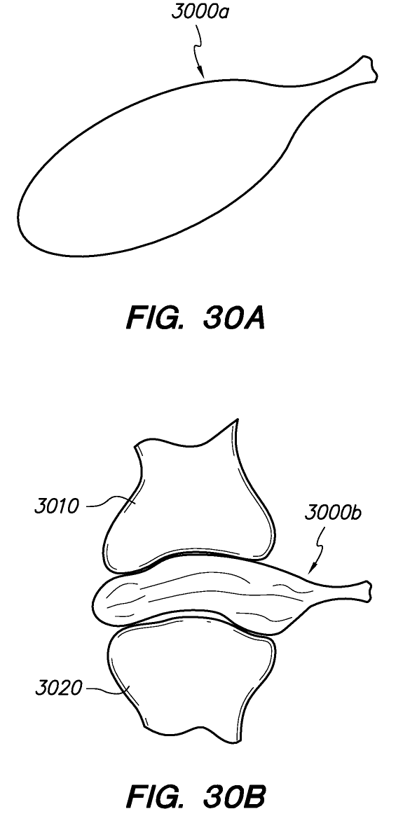

In one embodiment of an orthopedic delivery device system a balloon 3000a can be used to spread the bones of a joint apart as the delivery device is advanced into the joint. In the embodiment illustrated in FIGS. 30A and 30B a specially shaped balloon that can be configured for use with specific joints, such as finger joints, can be combined with any of the devices or systems described herein. The balloon 3000 (shown in one embodiment at least partially inflated in 3000a and deflated in 3000b) can be a high pressure balloon and can be delivered through a cannula or needle, or be attached to the end of a needle. The balloon 3000b can be inserted between the bones and inflated using traditional angioplasty techniques. In one embodiment a spoon or football shape would allow for easy insertion through a large bore needle as intended for the procedure. Once inflated the balloon 3000a would or could assume the unique shape of the bones and could stretch the joint capsule. In one embodiment the balloon 3000 could be of a small profile when inserted, and in one example around 5 French (or about 0.067 inches or 1.67 mm in diameter) and inflate or blow up to 15 French (or about 0.197 inches or 5 mm in diameter) or so. In one embodiment the balloon 3000 can be pleated or folded. In one embodiment the balloon 3000 can be removed or remain in place during device delivery, and can undergo partial or complete inflation or deflation during the device delivery.

In one embodiment of an orthopedic device delivery system a sizing template for knuckle evaluation and device size can be used.

One embodiment of a of an orthopedic device delivery system comprises a delivery handle and a loading device that straighten an implantable device or implant, then eject the implantable device or implant in a controlled and defined plane into a joint of the body of a patient. An embodiment of a delivery handle is configured to advance a plunger (or push rod or advance rod) as previously described herein, and is shaped so that it the orientation of any of the embodiments of delivery channels, needles, cannulae, and similar structures are directed for proper alignment and orientation for implantation or extraction. One embodiment of a loading device is configured to advance an orthopedic implant into the delivery channel, needle, cannula, or similar structures. For example, one embodiment of a cannula is configured to be fixed to a handle so that the cannula can not move with respect to the handle, thereby determining an orientation of the implant with the delivery device. In one embodiment, the cannula can be locked in place to be fixed with respect to a handle, and in another embodiment, the cannula can be permanently fixed to the handle.

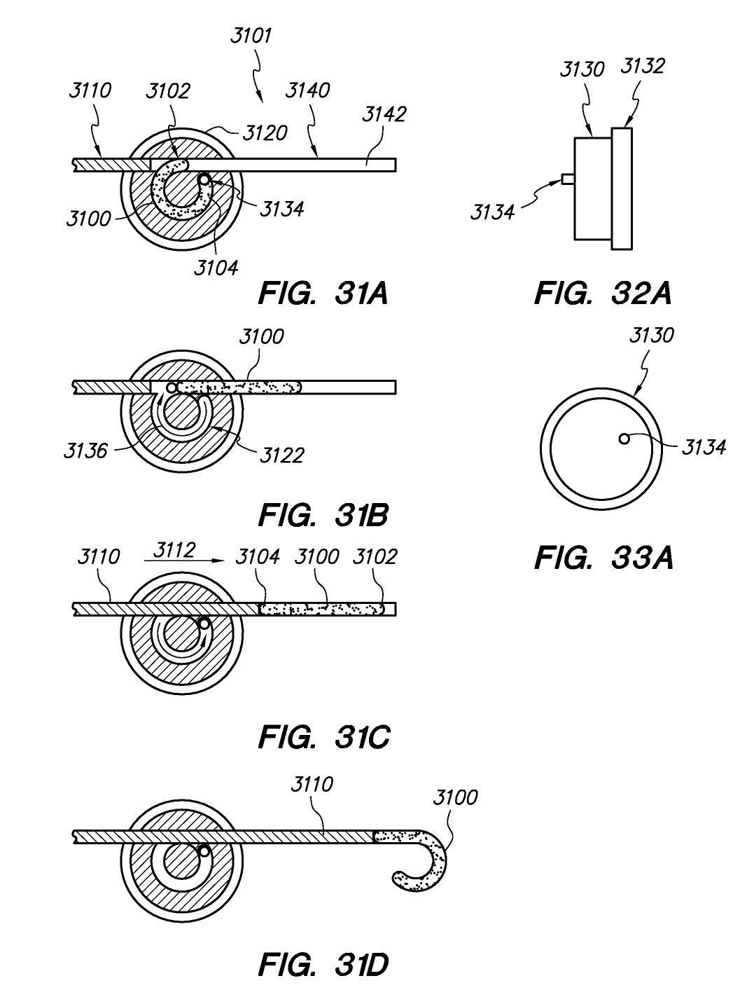

Various embodiments of an orthopedic device delivery system comprise a handle, delivery mechanism (also called a plunger, push rod, or advance rod) and a loading device (also called a device loader or cassette) that holds one or more orthopedic devices in a non-straightened configuration until the orthopedic device is straightened for delivery through the lumen of a cannula into the delivery site in the patient's body. Certain embodiments of this type of orthopedic device delivery system are illustrated in FIGS. 31A-41C. The handle, which can be held by a medical practitioner, provides for linear translation (forwards or backwards) of the plunger. In various non-limiting embodiments, the translation of the plunger can be accomplished by a number of different means, such as a rotating knob, trigger, indexing (similar to a mechanical pencil), hand gun, ratcheting type mechanism, screw type mechanism, and/or a rack and pinion. The delivery mechanism, or plunger, can be flexible or rigid, as previously disclosed above, and is configured to move an orthopedic device through a lumen in a cannula for delivery or retrieval to or from a patient's body. FIGS. 31A-41C illustrate various embodiments of orthopedic device delivery systems.

In one embodiment, as illustrated in FIGS. 31A-31D, an orthopedic device delivery system 3101 comprising a plunger 3110, a loading device 3120 (that can also be called a cassette) and a cannula 3140. The loading device 3120 comprises a storage delivery channel 3122 that contains and/or stores one or more orthopedic devices 3100 in a natural, non-straightened configuration. In one embodiment a natural configuration of an orthopedic device is arcuate, or a curved configuration. The proper orientation of the orthopedic device 3100 for delivery into a patient is assured by configuring the system 3101 such that the orthopedic device 3100 exits the cannula 3140 such that it is deployed in an orientation or plane substantially parallel to an articular bone surface in a joint. The orthopedic device 3100 comprises a distal end 3102 and a proximal end 3104. The orthopedic device delivery system 3101 straightens the orthopedic device 3100 for accurate delivery by assuring stability and proper orientation upon deployment. The loading device 3120 can be single use, disposable, or re-usable. The loading device 3120 is removably attachable to any previously described delivery or retrieval system, including embodiments with plungers, etc. or with any embodiment described in conjunction with FIGS. 10A-15C and 21A-30B. As illustrated in FIGS. 31A-31D, the embodiment of the loading device 3120 is fixed to a cannula 3140 using any attachment configurations, such as permanent fixation, bonding, a snap fit, lock, threaded engagement, form fit, bonding, or mechanical locking mechanism to fix the orientation of the cannula 3140 with respect to the loading device 3120 for proper alignment and orientation for delivery or retrieval of an orthopedic device 3100. In one embodiment, the loading device 3120 comprises an advancement mechanism, such as by spring loading or manual advancement, such as using a knob 3130 (one embodiment is illustrated in alternate views in FIGS. 32A and 33A) to advance the orthopedic device 3100. One embodiment of a knob 3130 comprises an actuation surface 3132 for rotation or manipulation to move the knob 3130 and a delivery pin 3134. The delivery pin 3134 is fixed to the knob 3130. Rotation or actuation of the knob 3130 moves the delivery pin 3134 within the delivery channel 3122 to move the proximal end 3104 of the orthopedic device 3100 into a lumen 3142 of the cannula 3140. In the illustrated embodiment, rotation of the knob 3130 moves the delivery pin 3134 in a clockwise motion indicated by arrow 3136.

FIG. 31A illustrates the device in its loaded, normal state. In various embodiments, the distal end 3102 of the orthopedic device 3100 can rest within the delivery channel 3122 or protrude in to the lumen 3142 of the cannula 3142. Operation of the loading device 3120 brings the orthopedic device 3100 from the delivery channel 3122 into a position to be actuated by the plunger 3110 through the lumen 3142 of the cannula 3140 for delivery into the patient. The delivery channel 3122 is in communication with the lumen 3142 of the cannula 3140. The distal 3102 or leading end of the orthopedic device 3100 is positioned in or near the entrance to the lumen 3142 of the cannula 3140 and the proximal end 3104 rests against the delivery pin 3134.

When the knob 3130 is rotated or actuated as illustrated in FIGS. 31B-31C, the orthopedic device 3100 is loaded into the lumen 3142 of the cannula 3140 in a specific, proper orientation, and changes configuration to a more straightened form inside the lumen of the cannula 3140. The various embodiments of loading devices can be used with any of the embodiments of the orthopedic devices described herein. As the knob 3130 is rotated clockwise it forces the delivery pin 3134 against the proximal end 3104 and pushes the orthopedic device 3100 clockwise through the delivery channel 3122 to the lumen 3142 of the cannula 3140 where the orthopedic device 3100 is straightened. The orthopedic device 3100 is moved from its normal arcuate configuration in to a substantially straightened configuration while maintaining its proper orientation for delivery. The knob 3130 can be spring loaded to return to its initial position when the actuation surface 3132 is released. FIGS. 31C and 31D illustrate the plunger 3110 being advanced distally along the arrow marked 3112 within the lumen 3142 of the cannula 3140 to advance the orthopedic device 3100 out of the cannula 3140 and into the patient in an orientation for proper delivery. One embodiment of the orthopedic device delivery system 3101 provides for the orthopedic device 3100 to exit the cannula 3140 in a plane that is substantially the same as the original loaded state within the delivery channel 3122 to help assure proper deployment orientation. As the orthopedic device 3100 exits the lumen 3142 of the cannula 3140 the orthopedic device 3100 can return to its arcuate configuration, in a clock wise direction in a plane that is substantially the same or parallel to the plane of the delivery channel 3122 within the a loading device 3120. With the proper orientation of the orthopedic device delivery system 3101, the orthopedic device 3100 can be delivered in a plane substantially parallel to a plane between the articulating bony or cartilaginous surfaces within a joint.

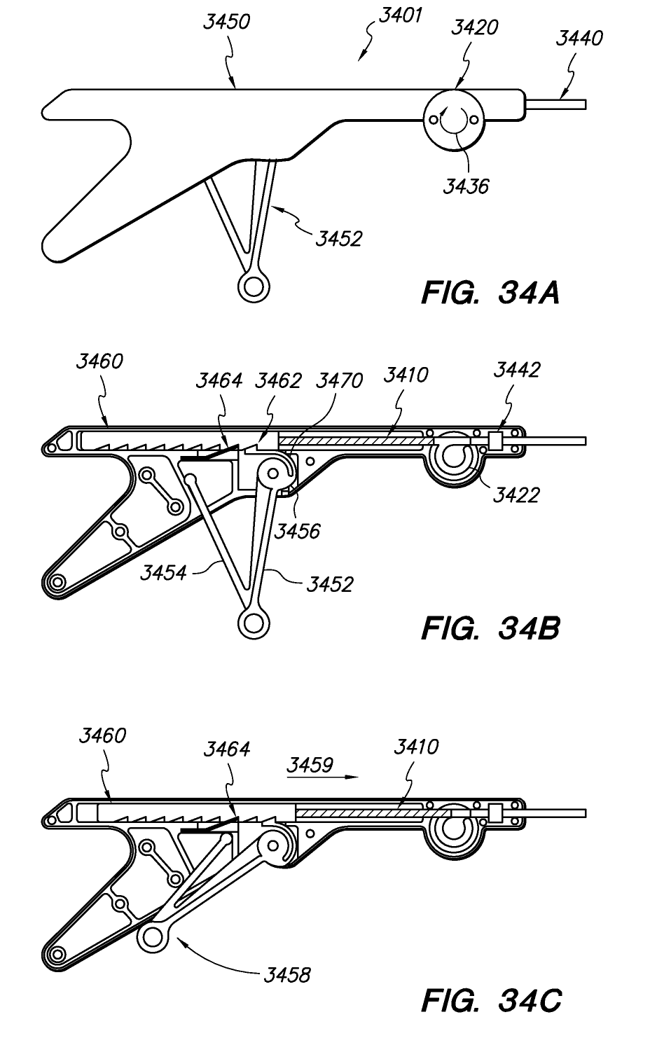

In one embodiment, as illustrated in FIGS. 34A-34C, an orthopedic device delivery system 3401 similar to orthopedic device delivery system 3101 comprises a cannula 3440, a loading device 3420 with delivery channel 3422 and a handle 3450 with a pistol grip configuration. The handle 3450 comprises a trigger 3452 for advancing the plunger 3410 with a rack and pinion linear advancement mechanism. In one embodiment the one-piece, unitary trigger 3452 comprises a trigger head 3456 and a trigger return 3454. Movement of the trigger 3452 in the direction indicated by arrow 3458 engages the rack 3460. The rack 3460 comprises at least a first ratchet head 3462 and additional ratchet heads, or teeth. A ratchet 3464 is configured to engage the ratchet heads 3462 along the rack 3460 to prevent the rack 3460 from moving backwards during advancement of the plunger 3410. The trigger 3452 engages the rack 3460 at the first ratchet head 3462. When the trigger 3452 is pulled back the ratchet arm 3470 winds down around the trigger head 3456 and pulls the ratchet tooth 3462 along with the rack 3460 distally in the direction of arrow 3459 to linearly advance the plunger 3410. The construction of the ratchet arm 3470 to trigger head 3456 is such that the cantilever provides a spring force and resists downward deflection. Upon release of the trigger 3452 the ratchet arm 3470 releases it's built up energy and returns the trigger 3452 to a neutral position (forward as illustrated in FIGS. 34A and 34B). As the trigger 3452 returns the ratchet head 3462 advances distally along arrow 3459 and the ratchet arm 3470 biases down until the ratchet arm 3470 can spring back into the next tooth, or ratchet head 3462. In one embodiment, the cannula 3440 is locked to the handle 3450 with a cannula lock 3442. In one embodiment, the cannula 3440 and cannula lock 3442 are a unitary, single body.

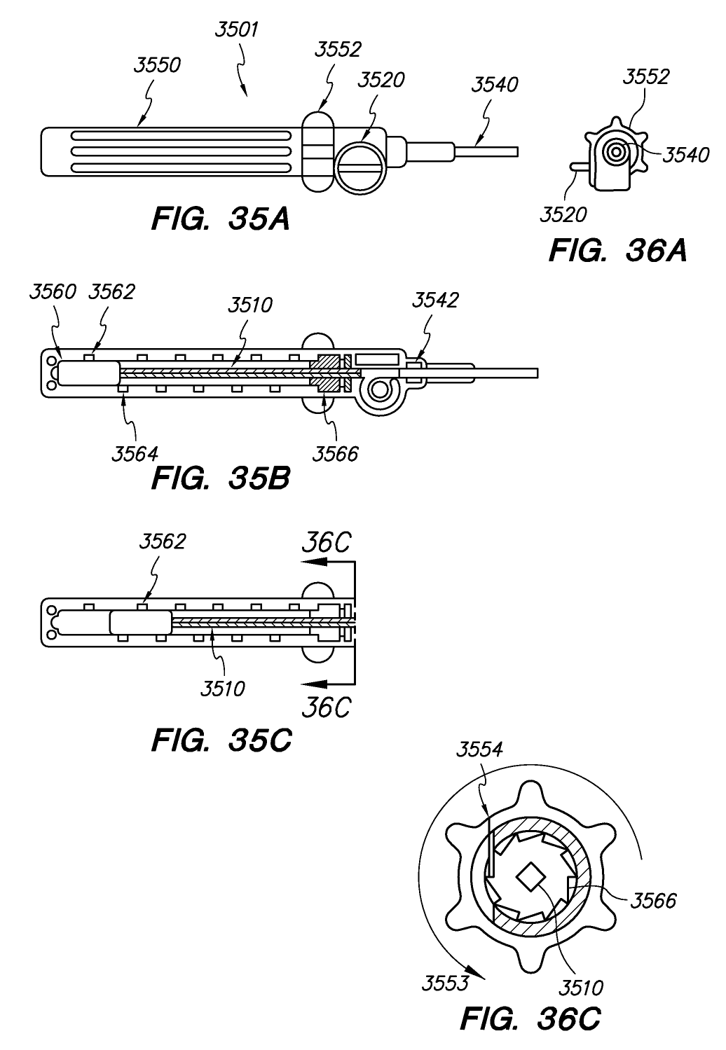

In one embodiment, as illustrated in FIGS. 35A-35C and FIG. 36A, an orthopedic device delivery system 3501 similar to orthopedic device delivery system 3101 comprises a cannula 3540, a loading device 3520, a delivery knob 3552 and a handle 3550. The cannula 3540 is similar to previously described cannulae, and in the embodiment illustrated includes a cannula lock 3542. The loading device 3420 is similar to embodiments of the loading device knob described above. The delivery knob 3552 works with a follower 3560 and a follower pin 3562. In one embodiment the follower 3560 is attached to a non-circular cross-section push rod 3510. In one embodiment the push rod 3510 has a square cross section, but it can have any cross sectional shape that allows it to rotationally engage the inside of the delivery knob 3552 while still free to slideably actuate or move axially through the axis of the delivery knob 3552. Rotation of the delivery knob 3552 rotates the push rod 3510. In one embodiment, clockwise rotation as viewed in FIG. 36A or 36C causes the follower pin 3562 to move through a helical track 3564 disposed inside the handle 3550 around the follower 3560. The rotational motion of the delivery knob 3552 rotates the push rod 3510 which turns the follower pin 3562 in the helical track 3564 which results in axial movement of the follower 3560 and push rod 3510 to advance the orthopedic device out of the lumen of the cannula 3540.

FIG. 36C illustrates an embodiment of drive system for a follower 3560. The cross section of the push rod 3510 is square or some shape other than round. The push rod 3510 slides axially through the ratchet drive 3566. The ratchet drive 3566 can spin freely inside the handle 3550. The delivery knob 3552 comprises a ratchet pawl 3554 and is secured to the handle 3550 for rotational actuation. The ratchet pawl 3554 engages the ratchet drive 3566. As the delivery knob 3552 is turned in the direction of the arrow marked 3553, the rotation indexes the ratchet drive 3566 counter-clockwise as viewed from the end of the orthopedic device 3501 as illustrated in FIG. 36C. Since the push rod 3510 is square in this example and the hole through the axis of the ratchet drive 3566 is square as well, the rotation of the delivery knob 3552 also turns with the ratchet drive 3566. This causes the push rod 3510 to rotate as well. When the push rod 3510 rotates it twists the follower 3560 through the track 3564 via the follower pin 3562. Since the push rod 3510 is not axially fixed it moves distally as the follower 3560 is spun. The delivery knob 3552 is manually returned by twisting it in the opposite direction. This causes the ratchet pawl 3554 to disengage and subsequently fall into the next tooth in the ratchet drive 3566. The process can repeat itself until the follower 3560 abuts against the proximal end of the ratchet drive 3566.

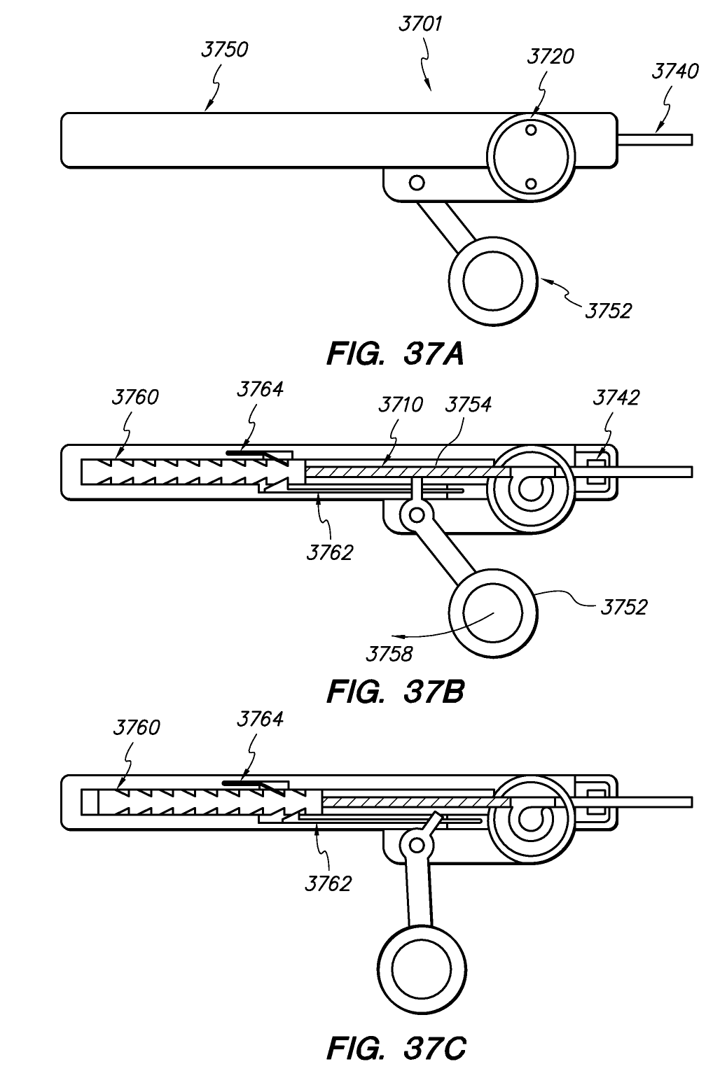

In one embodiment, as illustrated in FIGS. 37A-37C, an orthopedic device delivery system 3701 similar to orthopedic device delivery system 3101 comprises a cannula 3740, a loading device 3720, a handle 3750 and a finger-loop trigger 3752. The cannula 3740 is similar to previously described cannulae, and in the embodiment illustrated includes a cannula lock 3742. The handle 3750 comprises a trigger 3752 for advancing the push rod 3410 with a rack and pinion linear advancement mechanism. The rack 3760 has teeth on both sides as illustrated in FIGS. 37B and 37C. The secondary ratchet 3764 engages the top set of teeth and keeps the rack 3760 from sliding back when the primary ratchet 3762 is returned to its starting position. The trigger 3752 has a trigger pawl 3754 that engages the primary ratchet 3762. The primary ratchet 3762 has at least one through hole or slot for the trigger pawl 3754 to fit within. As the trigger 3752 is pulled back in the direction indicated by arrow referenced as 3758 the trigger 3752 causes the trigger pawl 3754 to pivot forward in the distal direction. As the trigger pawl 3754 pivots forward the trigger pawl 3754 pushes the slot in the primary ratchet 3762 forward in the distal direction. This in turn causes the primary ratchet 3762 to move forward and move the rack 3760 forwards. The secondary ratchet 3764 pops into the next tooth. When the trigger 3752 is moved to the forward or starting position it moves the primary ratchet 3762 backwards via the trigger pawl 3754 engagement into the next tooth.

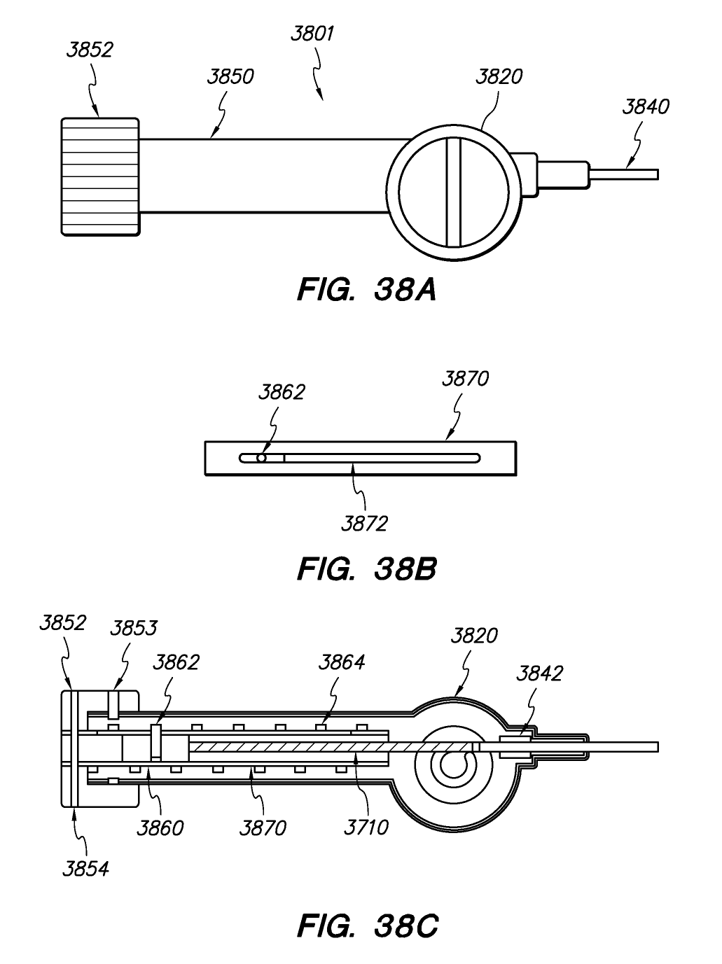

In one embodiment, as illustrated in FIGS. 38A-38C, an orthopedic device delivery system 3801 similar to orthopedic device delivery system 3101 comprises a cannula 3840, a loading device 3820, a proximal delivery knob 3852 and a handle 3850. The cannula 3840 is similar to previously described cannulae, and in the embodiment illustrated includes a cannula lock 3842. The loading device 3820 is similar to embodiments of the loading device knob described above. The proximal delivery knob 3852 is attached to a rotating advance tube 3870 that drives a follower 3860 through a track 3864. The advance tube 3870 has a slot 3872 along at least a portion of the length of the advance tube 3870. The slot 3872 has a width that is slightly oversized to the diameter of the follower pin 3862. Rotation in the delivery knob 3852 rotates the advance tube 3870, which pushes the follower pin 3862 through the helical track 3864 that runs along at least a length of an interior channel in the handle 3850. The follower pin 3862 is attached to the follower 3860, which is connected to the push rod 3810. Rotation of the delivery knob 3852 causes the follower 3860 to advance along the track 3864. The rotational motion of the delivery knob 3852 results in axial movement of the follower 3860 and push rod 3810 to advance the orthopedic device out of the lumen of the cannula 3840. The knob 3852 is attached to the advance tube 3870 with an advance lock 3854. In one embodiment the advance lock 3854 is a dowel pin. In one embodiment the knob 3852 can be secured to the body of the handle 3850 with the knob lock 3853, such as a dowel pin that rides in a groove in the body of the handle 3850. Rotating the knob 3852 in the opposite direction causes the follower pin 3862 to move proximally, or backwards, along the track 3864 causing the follower 3860 and the push rod 3810 to move proximally, or backwards, as well.

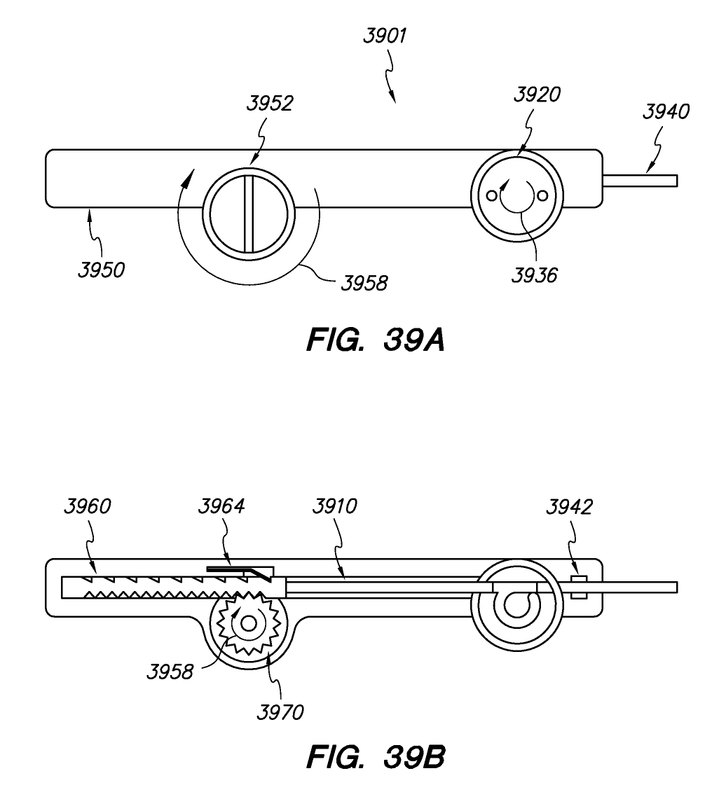

In one embodiment, as illustrated in FIGS. 39A-39B, an orthopedic device delivery system 3901 similar to orthopedic device delivery system 3101 comprises a cannula 3940, a loading device 3920, a delivery knob 3952 and a handle 3950. The cannula 3940 is similar to previously described cannulae, and in the embodiment illustrated includes a cannula lock 3942. The loading device 3920 is similar to embodiments of the loading device knob described above, and loads an orthopedic device into the cannula 3940 when the loading device 3920 is rotated in a direction indicated by the arrow referenced in FIG. 39A as 3936. In various embodiments the delivery knob 3952 can be positioned proximally and to a side of the orthopedic device delivery system 3901. In one embodiment the delivery knob 3952 is on the same side as the loading device 3920. The delivery knob 3952 provides axial movement to push the orthopedic device with a push rod 3910 through the cannula 3940 and in to a patient when the delivery knob 3952 is rotated in a direction indicated by the arrow referenced in FIG. 39A as 3958. The delivery knob 3952 engages the rack 3960 by way of a drive wheel 3970. The drive wheel 3970 has teeth that engage corresponding teeth on the rack 3960. A ratchet 3964 keeps the rack 3960 from moving backwards (or proximally) when the drive wheel 3970 is not turned. In one non-illustrated embodiment, the ratchet 3964 could be a part of the housing for the drive wheel 3970.

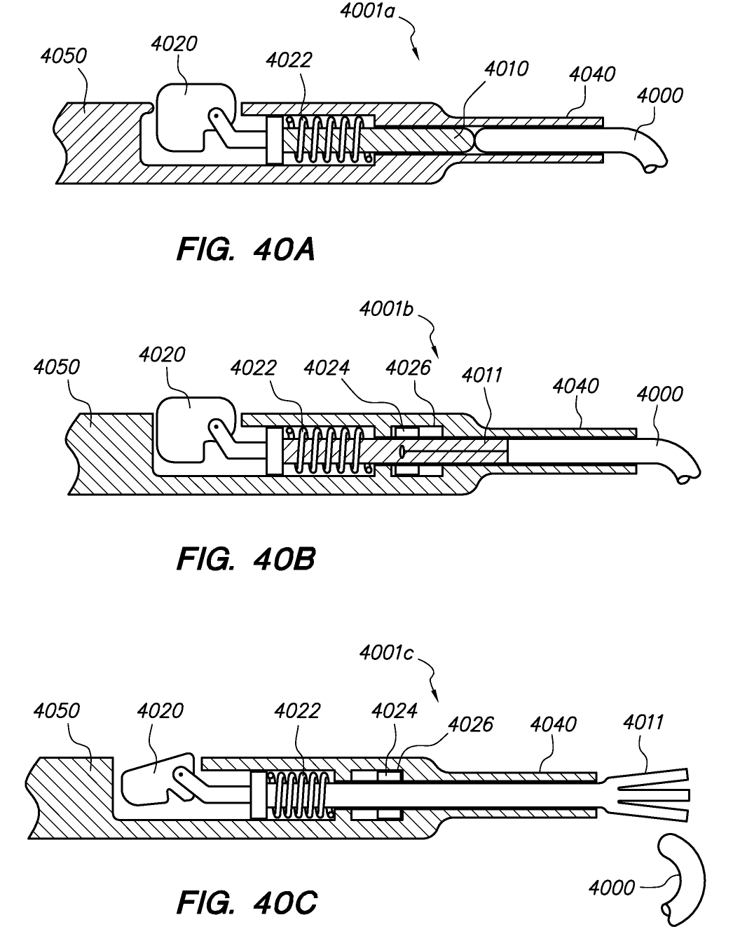

In one embodiment, as illustrated in FIG. 40A, an orthopedic device delivery system 4001 similar to orthopedic device delivery system 3101 comprises a cannula 4040, a handle 4050 and a push-button 4020 to axially advance an orthopedic device 4000 distally though the lumen of the cannula 4040 into a patient. The orthopedic device delivery system 4001 is configured for one-handed actuation, using a trigger-type mechanism. Another one-handed actuation can be with rotation of a knob with a finger or thumb, or actuation of a trigger with a single hand. In one embodiment an orthopedic device delivery system 4001a uses a mechanically actuated push rod 4010 that is in contact with the proximal end of the orthopedic device 4000 to advance the orthopedic device 4000 through the cannula 4040. In one embodiment of a push rod 4011, as illustrated in FIGS. 40B-40C in a delivery system 4000b and 4000c similar to the orthopedic device delivery system 4001a comprises a push rod 4011 connectable to an orthopedic device 4000 that can be moved proximally and/or distally though the lumen of the cannula 4040 into a patient. Although the push rod 4011 with a connection is illustrated with a push-button orthopedic device delivery system as shown with orthopedic device delivery system 4001a, it can be used with any orthopedic device delivery system herein. Although not illustrated in FIGS. 40A-40C the axial advancement mechanism can be used in conjunction with any of the embodiments of loading devices including loading knobs and cassettes described above. In one embodiment an orthopedic device delivery system 4001b comprises a push rod 4011 that includes a detachable connection between the push rod 4011 and the orthopedic device 4000, such as a releasable collet attachment for manipulation of the orthopedic device 4000 within the patient prior to release of the orthopedic device 4000. A spring 4022 may be used to return the push rod 4010 or 4011 to a proximal position. A sleeve 4024 in an opening 4026 can allow opening and closing of the collet attachment in 4011. Pressing the push button 4020 advances the orthopedic device 4000 distally along the cannula 4040. In one embodiment, the push rod 4011 is a collet attachment that allows for manipulation, positioning, repositioning, release, or re-capture and removal of the orthopedic device within the joint, if necessary. In one embodiment the push rod 4011 is releasably attachable to the proximal end of the orthopedic device and can release the orthopedic device or recapture it. In one embodiment, the sleeve 4024 can be actuated by the user with a switch or other mechanism to release the orthopedic device 4000 from the push rod 4011 by allowing the distal end of the push rod 4011 to expand when the sleeve 4024 is in a proximal position, as shown in FIG. 40B and FIG. 40C. When the sleeve 4024 is in a distal position (not illustrated here) the orthopedic device is held in the push rod 4011. In non-illustrated embodiments, the sleeve can be closer to the distal end of the push rod 4011.

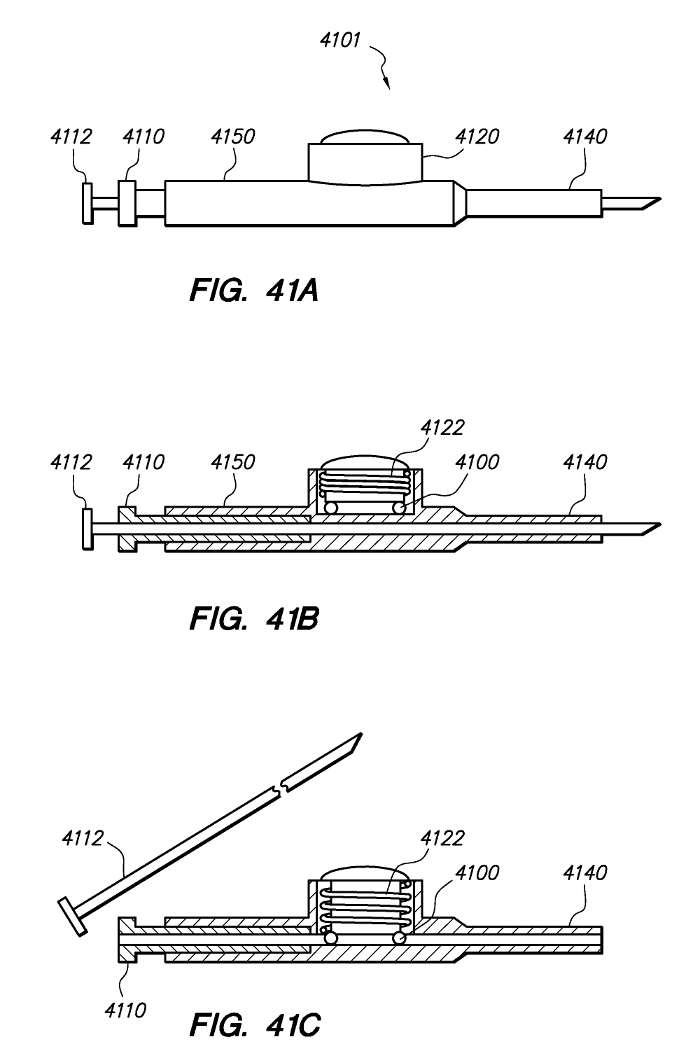

In one embodiment, as illustrated in FIGS. 41A-41C, an orthopedic device delivery system 4101 similar to orthopedic device delivery system 3101 comprises a cannula 4140, a handle 4150, a loading device 4120 and a removable tissue piercing device 4112. In one embodiment the loading device 4120 stores one or more orthopedic devices 4000 that can be actuated in line with the lumen of the cannula 4140 with a mechanism 4122, such as a spring, that will align the orthopedic device 4000 for delivery once a removable tissue piercing device 4112, such as a trocar (solid or tubular) is removed from the device after piercing tissue in a patient to access a delivery site. When the tissue piercing device 4112 is removed proximally out of the orthopedic device delivery system 4101 as shown in FIG. 41C, the loading device 4120 moves the orthopedic devices 4000 for loading into the cannula 4140.

It will be understood that the foregoing is only illustrative of the principles of the invention, and that various modifications, alterations, and combinations can be made by those skilled in the art without departing from the scope and spirit of the invention. Any of the embodiments of the various orthopedic devices disclosed herein can include features described by any other orthopedic devices or combination of orthopedic devices herein. For example, at least the following orthopedic device as indicated by reference numbers may have features that can be combined or interchanged with other orthopedic devices: at least 100a, 100b, 100c, 300a, 300b, 300c, 300d, 300e, 570d, 570e, 570f, 1100, 1200, 1600, 1700, 1800, 1900, 2000, 2120, 2200, 2600, 2700, 2700, 3100, 4000 and 4100. Furthermore, any of the embodiment of the various orthopedic device delivery and/or retrieval systems can be used with any of the orthopedic devices disclosed, and can include features described by any other orthopedic device delivery and/or retrieval systems or combination of orthopedic device delivery and/or retrieval systems herein. For example, at least the following orthopedic device orthopedic device delivery and/or retrieval systems as indicated by reference numbers may have features that can be combined or interchanged with other orthopedic device delivery and/or retrieval systems: at least 1000, 1801, 1901, 2001, 2210, 2310, 2800, 3101, 3401, 3501, 3701, 3801, 3901, 4001 and 4101. Accordingly, it is not intended that the invention be limited, except as by the appended claims.

What is claimed is:

1. An orthopedic device delivery system suitable for minimally invasive deployment of an orthopedic device in a directed orientation into a delivery site in a patient's body, comprising:
a tubular delivery apparatus with a lumen extending there through; and
a loading device comprising a channel in communication with the lumen of the tubular delivery apparatus, the channel configured to store at least one orthopedic device in a first configuration, the loading device being configured to move the at least one orthopedic device from the first configuration to a second intermediate delivery configuration wherein the orthopedic device is moveable through the lumen in a narrowed configuration, the loading device assuring the at least one orthopedic device will deploy out a distal end of the lumen in a third deployed configuration comprising a generally arcuate configuration at substantially body temperatures to enhance positioning of the orthopedic device when deployed.

2. The orthopedic device delivery system of claim 1, wherein the first configuration is substantially the same as the third deployed configuration.

3. The orthopedic device delivery system of claim 1, wherein the second intermediate delivery configuration is a narrowed configuration.

4. The orthopedic device delivery system of claim 1, wherein the second intermediate delivery configuration is a substantially straightened configuration.

5. The orthopedic device delivery system of claim 1, further comprising at least one orthopedic device comprising an elongate core having a proximal end and a distal end, the elongate core comprising a generally arcuate configuration at substantially body temperatures to enhance positioning of the orthopedic device when deployed, said elongate core being manipulatable into a substantially straightened configuration to permit delivery.

6. The orthopedic device delivery system of claim 1, wherein the loading device comprises a knob configured to move an orthopedic device from the channel of the loading device into the lumen of the tubular delivery apparatus.

7. The orthopedic device delivery system of claim 1, further comprising a plunger for advancing an orthopedic device out of the lumen of the tubular delivery apparatus and in to the patient.

8. A method of minimally-invasively deploying an orthopedic device in to an orthopedic joint in a patient, comprising:
moving an orthopedic device from a first configuration to a second configuration;
moving the orthopedic device from the second configuration to a third configuration subsequent to the step of moving the orthopedic device from the first configuration to the second configuration, the third configuration corresponding to the configuration of the orthopedic device as deployed in situ in an orthopedic joint, the second configuration controlling the orientation of the orthopedic device for proper deployment of the orthopedic device in the third configuration.

9. The method of minimally-invasively deploying an orthopedic device in to an orthopedic joint of claim 8, the first configuration being generally arcuate.

10. The method of minimally-invasively deploying an orthopedic device in to an orthopedic joint of claim 8, the second configuration being a narrowed configuration.

11. The method of minimally-invasively deploying an orthopedic device in to an orthopedic joint of claim 8, the second configuration being substantially straightened.

12. The method of minimally-invasively deploying an orthopedic device in to an orthopedic joint of claim 8, the third configuration being generally arcuate.

13. The method of minimally-invasively deploying an orthopedic device in to an orthopedic joint of claim 8, the third configuration being substantially similar to the first configuration.

14. The method of minimally-invasively deploying an orthopedic device in to an orthopedic joint of claim 8, wherein the orthopedic device comprises an elongate core having a proximal end and a distal end, the core comprising a generally arcuate configuration at substantially body temperatures to enhance positioning of the orthopedic device when deployed, said core being manipulatable into a substantially straightened configuration to permit delivery; and an articular layer surrounding at least a portion of the core.

15. The method of minimally-invasively deploying an orthopedic device in to an orthopedic joint of claim 8, wherein an orthopedic delivery system comprising a loading device stores at least one orthopedic device in the first configuration.

16. The method of minimally-invasively deploying an orthopedic device in to an orthopedic joint of claim 8, further comprising attaching the articular layer to at least a portion of the core during implantation in the patient.

17. The method of minimally-invasively deploying an orthopedic device in to an orthopedic joint of claim 8, further comprising attaching the articular layer to at least a portion of the core after implantation in the patient.

18. The method of minimally-invasively deploying an orthopedic device in to an orthopedic joint of claim 8, further comprising inserting an apparatus at or proximal the situs of deployment to remove at least a portion of the core.

19. An orthopedic device suitable for minimally invasive deployment using a tubular delivery apparatus, the orthopedic device comprising:
a biocompatible outer surface;
a coating disposed on at least a part of the outer surface; and
an elongate core having a proximal end and a distal end, the core comprising a generally arcuate configuration at substantially body temperatures to enhance positioning of the orthopedic device when deployed, said core being manipulatable into a substantially straightened configuration to permit delivery.

20. The orthopedic device of claim 19, wherein the biocompatible outer surface comprises a biological covering.

21. The orthopedic device of claim 19, wherein the biocompatible outer surface comprises material configured to increase from a first dimension to a second, larger dimension after implantation.

22. The orthopedic device of claim 19, wherein the coating is porous.

23. The orthopedic device of claim 19, wherein the coating elutes a medium.

24. The orthopedic device of claim 23, wherein the medium is a drug.

25. The orthopedic device of claim 19, wherein the biocompatible outer surface comprises an outer surface of the elongate core.

26. The orthopedic device of claim 19, further comprising an articular layer surrounding at least a portion of the core, wherein the biocompatible outer surface comprises an outer surface of the articular layer.

27. The orthopedic device of claim 26, wherein the device is further configured to permit at least partial removal of at least part of the core from the articular layer resulting in a void within the articular layer.

28. The orthopedic device of claim 27, wherein the device is further configured so that at least part of the void created by removal of at least part of the core is partially filled with a polymer material.
